(19) United States
(12) Patent Application Publication
(10) Pub. No.: US 2026/0165934 A1
(43) Pub. Date: Jun. 18, 2026

(54) COMPOSITIONS AND METHODS FOR LIGHTENING HAIR

(71) Applicant: L'Oreal, Paris (FR)

(72) Inventors: Tathagata MUKHERJEE, Union, NJ (US); Martin ASARE, Springfield, NJ (US); Siva Muthukrishnan SIVARAMAKRISHNAN, Bridgewater, NJ (US)

(21) Appl. No.: 18/986,145

(22) Filed: Dec. 18, 2024

(51) Int. Cl. A61K 8/46; A61Q 5/08

(52) U.S. Cl. CPC A61K 8/463; A61Q 5/08; A61K 2800/882; A61K 2800/884

(57) ABSTRACT

The disclosure relates to compositions and methods for lightening the color of hair. The compositions and methods can provide a very high level of lift while minimizing damage to the hair.

### TECHNICAL FIELD

The present disclosure relates to compositions for lightening hair, kits comprising the compositions, and methods for using the compositions.

### BACKGROUND

It is known that consumers desire to use cosmetic and care compositions that enhance the appearance of keratinous substrates such as hair, e.g., by changing the color of the hair, and/or by imparting various properties to hair, such as shine and conditioning. The process of altering the color of hair may, for example, involve lifting the color of the hair to lighten the hair color, and/or depositing an artificial color onto the hair which provides a different or darker shade or color to the hair. The process of lifting the color of hair, also known as lightening, generally requires the use of compositions that comprise oxidizing agents. Such oxidizing agents enter the hair shaft and react with melanin in the hair, thereby lightening the color of the hair.

Variation of tone height before and after the application of a lightening composition is used to evaluate lightening or lifting of the color of the hair. Degree or level of lightening or lift corresponds to the variation. The notion of “tone” is based on the classification of the natural shades, one tone separating each shade from the shade immediately following or preceding it, which is well known to hairstyling professionals. Tone heights or levels range from 1 (black) to 10 (light blonde) or even up to 12 (platinum), one unit corresponding to one tone. Thus, the higher the number, the lighter the shade or the greater the degree of lift.

Many consumers seek high degrees of lift, such as when going from black to light blonde. The level of lift desired is typically achieved by using varying amounts of oxidizing and/or alkaline agents in the lightening composition, or by varying the length of time the composition is left on the hair. In cases where very high levels of lift are sought (e.g. at least 6), multiple applications of the lightening composition are often required to achieve the desired shade.

Additionally, consumers also often seek tones that are lighter and brighter. This can be achieved by altering the undertones of the hair color. Typically, undertones are altered or neutralized by applying a separate toning composition to the hair after the hair is lightened, for example to make the tone appear warmer or cooler, get rid of brassy undertones, etc.

However, compositions for lightening and toning hair are known to be harsh and to damage the hair fibers and/or irritate the scalp during the lightening treatment. Compositions and processes that are used for high (e.g. at least 6) levels of lift are particularly damaging due to the need for increased amounts of harsh chemicals to achieve the desired lightening effect and for increased processing times, meaning that the damaging compositions are in contact with the hair fibers for a longer period of time. In addition, sometimes it is necessary to perform the lightening process more than once, especially when high or very high levels of lift are desired. Such multiple processing applications are also particularly damaging to hair. All of these treatments therefore lead to hair that feels rough, tangles easily, and looks unhealthy. Thus, in order to reduce or avoid damage to the hair, as well as to improve the cosmetic performance of these compositions, new lightening compositions and/or processes for achieving the desired level of lift, preferably in less time, with fewer applications, and/or with reduced amounts of harsh chemicals, are needed.

Lightening compositions typically comprise persulfates which accelerate the speed of the hair lightening process. On the one hand this is advantageous because the harsh chemical treatment can be removed from the hair after a relatively shorter period of time, thus permitting less exposure of the hair fibers to the damaging compositions. On the other hand, however, persulfates themselves are a contributing cause of hair damage. Moreover, the reduced processing time must also be balanced against the amount of time needed to achieve the desired level of lift, because if the composition is removed too soon, the active agents will not have had sufficient time to de-colorize the hair fibers and the desired level of lightening will not be achieved.

There have been various attempts to mitigate hair damage during the lightening process. For example, some bleaching compositions contain additional components intended to reduce the damage to the hair fiber. However, this approach poses difficulties insofar as some components cause the compositions to be unstable, are detrimental to cosmetic attributes of the treated hair, and/or present other problems such as lack ease and uniformity of application, poor rheology or viscosity of the compositions, and/or reduced efficacy in lifting or lightening the color of the hair. Other compositions have been modified to remove certain damaging components, but these compositions are not able to achieve the desired level of lightening in the time the compositions can be left on the hair before damage occurs. It would, therefore, be desirable to provide compositions and methods that can lift the color of hair in an effective and expeditious manner, while leaving the hair with satisfactory cosmetic properties.

It has now been surprisingly discovered that hair lightening compositions according to the disclosure having reduced amounts of certain harsh chemicals such as persulfate compounds and oxidizing agents can be prepared which permit effective and efficient lightening, while also providing a smooth feel and healthy appearance of the treated hair.

### SUMMARY

The present disclosure relates to compositions and methods for lightening or lifting the color of the hair, which are able to achieve very high or even ultra-high levels of lift, even with lower amounts of persulfates and oxidizing agents. Damage during the lightening/bleaching process is minimized, which protects the integrity of the hair and leaves the hair stronger, softer, smoother, and with a healthier appearance than hair lightened with traditional hair bleaching compositions.

The disclosure relates to base lightening compositions which comprise reduced amounts of harsh chemicals such as persulfate compounds. The base lightening compositions comprise (i) at least one persulfate compound, for example at least two persulfate compounds, and (ii) at least one silicate compound. The total amount of (i) persulfate compounds may vary, but is advantageously less than about 50%, such as less than about 45%, or less than about 40%, for example may range from about 10% to not greater than 50% or from about 25% to about 45% by weight, relative to the total weight of the base lightening composition. In some embodiments, the base lightening compositions comprise (i) (a) ammonium persulfate and (b) at least one additional persulfate compound such as potassium persulfate and/or sodium persulfate. It has been discovered that choosing amounts of persulfate compounds relative to each other, very high levels of lift can be achieved in the same or even less time than with traditional base lightening compositions, leading to reduced damage to the hair. For example, in various embodiments the base lightening compositions may comprise ammonium persulfate and at least one additional persulfate compound, where the weight ratio of the amount of (a) ammonium persulfate to the total amount of (b) additional persulfate compounds is equal to or less than about 1, for example ranges from about 0.1 to about 0.9, such as from about 0.2 to about 0.8, from about 0.3 to about 0.7, or from about 0.4 to about 0.6.

The total amount of (ii) silicate compounds may vary, but generally ranges from about 5% to about 50%, such as from about 15% to about 45%, or from about 25% to about 40% by weight, based on the total weight of the base lightening composition. Optionally the base lightening compositions comprise at least one metasilicate, for example sodium metasilicate. In some embodiments, the base lightening compositions comprise at least two silicate compounds where at least one is a metasilicate, for example sodium silicate and sodium metasilicate.

The base lightening compositions may further comprise additional components such as alkalizing agents, thermal control agents, oxidizing agents, organic acids and their salts, oils, fillers, thickening agents, anionic surfactants, amino acids and their salts, and auxiliary components such as preservatives, hair conditioning agents, vitamins, chelating agents, pigments or colorants to color the composition, or any other component typically used in hair color lightening base compositions. Useful alkalizing agents include both organic and mineral alkalizing agents. It may in various embodiments be advantageous to include organic acids and their salts and/or amino acids and their salts to increase the integrity of the treated hair fibers. Useful thermal control agents may include urea, sodium stearate, and/or mineral oil. Thermal control agents that can be used may, in various embodiments, permit the hair lightening composition to achieve a peak temperature that is less than 70° C., for example less than about 65° C., less than about 63° C., less than about 60° C., less than about 58° C., less than about 55° C., less than about 53° C., or less than about 50° C.

In other embodiments the disclosure relates to base lightening mixtures, which are formed by mixing or combining a base lightening composition according to the disclosure with a solvent, for example water. By mixing or combining the base lightening composition with a solvent prior to or at the same time as mixing the base composition with an oxidizing agent, the risk of damage to the hair or scalp is reduced. In various embodiments, the amount of solvent that can be mixed with or added to the base lightening composition may range from about 1% to about 25%, such as from about 5% to about 20% by weight, relative to the total weight of the base lightening composition. In some embodiments, the solvent is added to the base lightening composition, or mixed together with the base composition and oxidizing agent, so that a weight ratio of total persulfates in the base lightening composition to the amount of solvent added ranges from about 1:1 to about 5:1, such as from about 1:1 to about 4:1, or from about 1.5:1 to about 3.5:1. In still other embodiments, the amount of solvent that can be mixed with or added to the base lightening composition may be chosen to provide a weight ratio of the total weight of the base lightening composition to the total weight of solvent ranging from about 5:1 to about 15:1, such as from about 6:1 to about 14:1, or about 7:1 to about 13.5:1.

The disclosure further relates to methods of preparing hair lightening compositions comprising mixing or combining (a) a base lightening composition, (b) solvent, and (c) oxidizing agent, as well as to hair lightening compositions prepared accordingly. Optionally, the (a) base lightening composition and (b) solvent may first be mixed to form a base lightening mixture, and subsequently the base lightening mixture and (c) oxidizing agent may be mixed to form a hair lightening composition according to the disclosure. For example, in various embodiments the mix ratio of [base lightening mixture]:[oxidizing composition] can range from about 1:3 to about 3:1, such as from about 1:2.5 to about 2.5:1, from about 1:2 to about 2:1, or from about 1:1.5 to about 1.5:1, or may be about 1:1. In other embodiments, however, the (a) base lightening composition, (b) solvent, and (c) oxidizing agent may all be mixed together at approximately the same time.

In other embodiments, instead of combining the base lightening composition with a solvent, an additional amount of an aqueous oxidizing composition can be used to form a hair lightening composition. For example, rather than adding from about 1% to about 25% of a solvent to the base composition, the amount of aqueous oxidizing composition mixed with the base lightening composition can be increased by about 1% to about 25%, all weights relative to the total weight of the base lightening composition. By way of example, the additional amount of aqueous oxidizing composition may be chosen so that a weight ratio of total persulfates in the base lightening composition to the additional amount of aqueous oxidizing composition ranges from about 1:1 to about 5:1, such as from about 1:1 to about 4:1, or from about 1.5:1 to about 3.5:1. In still other embodiments, the additional amount of aqueous oxidizing composition can be chosen to provide a weight ratio of the total weight of the base lightening composition to the total weight of additional aqueous oxidizing composition ranging from about 5:1 to about 15:1, such as from about 6:1 to about 14:1, or about 7:1 to about 13.5:1. The disclosure thus further relates to methods of preparing hair lightening compositions comprising mixing or combining (a) a base lightening composition, and (b) an oxidizing composition comprising an additional amount of aqueous oxidizing composition, as well as to hair lightening compositions prepared accordingly.

In further embodiments, a combination of solvent and aqueous oxidizing composition can be used.

In still further embodiments, a base lightening composition according to the disclosure is mixed with an oxidizing composition without the addition of solvent or additional oxidizing composition.

The disclosure also relates to methods of lightening the color of hair using hair lightening compositions according to the disclosure, in particular method for achieving very high or even ultra-high levels of lift.

In further embodiments still, the disclosure relates to kits comprising at least two, for example at least three, compartments or containers, wherein a first compartment or container comprises a base lightening composition according to the disclosure. Additional compartment(s) or container(s) may, for example, comprise an oxidizing composition or oxidizing agent, a solvent for mixing with the base lightening composition to prepare the base lightening mixture, an implement to mix the base composition and an oxidizing composition or oxidizing agent and/or to apply the mixture to the hair, or one or more additional hair care compositions, for example comprising a pre- and/or post-treatment hair conditioning composition.

### BRIEF DESCRIPTION OF THE FIGURES

FIG. 1A is a graph showing the ΔL values of hair treated with a comparative hair lightening composition and hair lightening compositions according to the disclosure at different strengths (volume) of developer.

FIG. 1B shows images of hair treated with a comparative hair lightening composition and hair lightening compositions according to the disclosure at different strengths (volume) of developer.

FIG. 2 is a graph showing the ΔL of hair treated with comparative hair lightening compositions and hair lightening compositions according to the disclosure at different strengths (volume) of developer over time.

FIG. 3 is a plot showing the ΔL and Δb of hair treated with comparative hair lightening compositions and hair lightening compositions according to the disclosure.

FIG. 4 is a plot showing the ΔL and Δb values of hair treated with comparative hair lightening compositions and hair lightening compositions according to the disclosure at different strengths (volume) of developer, for the same amount of time.

FIG. 5 shows images of hair treated comparative hair lightening compositions and hair lightening compositions according to the disclosure.

FIG. 6A shows SEM images of hair fibers treated with a comparative hair lightening composition after processing for 35 (S1), 50 (S2), or 70 (S3) minutes, at different magnifications.

FIG. 6B shows SEM images of an untreated hair fiber (S4), a hair fiber treated with a comparative hair lightening composition (S5), and hair fibers treated with hair lightening compositions according to the disclosure (S6-S7), at different magnifications.

FIG. 7 is a graph showing the ΔE of hair treated with comparative hair lightening compositions and hair lightening compositions according to the disclosure at different strengths (volume) of developer with either one or two applications, for the same amount of time.

FIG. 8 is a plot showing the ΔL and Δb values of hair treated with a comparative hair lightening composition and hair lightening compositions according to the disclosure.

FIG. 9 is a plot showing the ΔL and Δb values of hair treated with comparative hair lightening compositions and hair lightening compositions according to the disclosure.

### DETAILED DESCRIPTION

The disclosure relates to compositions that are useful for lightening or lifting the color of the hair, methods of lightening or lifting the color of the hair using the compositions, and kits that can be used in the methods.

### I. Compositions

It has been surprisingly and unexpectedly discovered that hair lightening compositions according to the disclosure provide very high or even ultra-high levels of lift while minimizing the damaging effects of bleaching and/or of oxidizing agents in the compositions.

Hair lightening compositions according to the disclosure can be prepared by mixing, at or near the time of use, a base lightening composition according to the disclosure with a solvent to form a base lightening mixture, and combining the base lightening mixture with an oxidizing composition or oxidizing agent to form a hair lightening composition. Hair lightening compositions according to the disclosure can also be prepared by mixing, at or near the time of use, a base lightening composition according to the disclosure with an oxidizing composition to form a hair lightening composition, where the amount of oxidizing composition is increased, for example by about 1% to about 25%, such as from about 5% to about 20% by weight, relative to the total weight of the base lightening composition.

### Base Lightening Compositions

Base lightening compositions according to the disclosure include at least one persulfate compound, for example at least two persulfate compounds, one or more silicate compounds, and optionally one or more compounds chosen from alkalizing agents other than persulfates, oxidizing agents, thermal control agents, organic acids and their salts, amino acids, amino sulfonic acids, oils, anionic surfactants, fillers, thickeners, or combinations of two or more thereof.

The base lightening compositions may be in various forms, including but not limited to powder, cream, lotion, or gel. In at least some embodiments, the base lightening compositions are anhydrous or substantially anhydrous, for example comprise less than 3%, such as less than 2%, less than 1.5%, less than 1%, less than 0.5%, less than 0.1%, or less than 0.01% water by weight, relative to the total weight of the base lightening composition. In some embodiments, the base lightening compositions are substantially or entirely pulverulent.

### Persulfate Compounds

Base lightening compositions according to the disclosure comprise at least one persulfate compound, for example at least two persulfate compounds. In a particular embodiment, the base lightening compositions comprise (a) ammonium persulfate, and (b) at least one additional persulfate compound, for example potassium persulfate and/or sodium persulfate.

The total amount of persulfate compounds in base lightening compositions according to the disclosure is less than found in typical base lightening compositions, but surprisingly hair lightening compositions according to the disclosure are just as effective, and in some cases more effective, at lifting or lightening the color of the hair, even when very high or ultra-high levels of lift are desired. In various embodiments, the base lightening compositions will comprise less than about 50% persulfate compounds, such as no more than 50%, no more than 49%, no more than 48%, no more than 47%, no more than 46%, or no more than 45% persulfate compounds by weight, relative to the total weight of the base lightening composition. For example, base lightening compositions according to the disclosure typically comprise a total amount of persulfate compounds ranging from at least about 10%, such as at least about 15%, at least about 20%, at least about 25%, at least about 30%, at least about 35%, or at least about 40%, but less than about 50%, or less than about 45% by weight, relative to the total weight of the base lightening composition, including all ranges and subranges thereof. By way of non-limiting example, the total amount of persulfate compounds in the base lightening compositions may range from about 10% to about 50%, about 10% to about 45%, about 10% to about 40%, about 10% to about 35%, about 10% to about 30%, about 10% to about 25%, about 15% to about 50%, about 15% to about 45%, about 15% to about 40%, about 15% to about 35%, about 15% to about 30%, about 15% to about 25%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, relative to the total weight of the base lightening composition. As further examples, the total amount of persulfates may range from about 30% to about 40%, such as from about 31% to about 40%, from about 32% to about 40%, from about 33% to about 40%, about 30% to about 39%, from about 31% to about 39%, from about 32% to about 39%, from about 33% to about 38%, about 30% to about 38%, from about 31% to about 38%, from about 32% to about 38%, from about 33% to about 38%, about 30% to about 37%, from about 31% to about 37%, from about 32% to about 37%, or from about 33% to about 37% by weight, relative to the total weight of the base lightening composition. In some embodiments, the total amount of persulfates may be about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight, relative to the total weight of the base lightening composition, including all ranges and subranges using any of the foregoing as upper and lower limits.

In embodiments where the base lightening composition comprises (a) ammonium persulfate and (b) at least one additional persulfate compound, the total amount of (a) ammonium persulfate in the base lightening composition can range from at least about 5%, such as at least about 6%, at least about 7%, at least about 8%, at least about 9%, at least about 10%, but less than about 20%, less than about 19%, less than about 18%, less than about 17%, less than about 16%, or less than about 15% by weight, relative to the total weight of the base lightening composition, including all ranges and subranges thereof. In some embodiments, the total amount of (a) ammonium persulfate may be about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the base lightening composition, including all ranges and subranges using any of the foregoing as upper and lower limits.

In embodiments where the base lightening composition comprises (a) ammonium persulfate and (b) at least one additional persulfate compound, the total amount of (b) additional persulfate compounds in the base lightening composition can range from at least about 15%, such as at least about 16%, at least about 17%, at least about 18%, at least about 19%, at least about 20%, at least about 22%, at least about 23%, at least about 24%, or at least about 25%, but less than about 40%, less than about 35%, or less than about 30% by weight, relative to the total weight of the base lightening composition, including all ranges and subranges thereof. In some embodiments, the total amount of (b) additional persulfate compounds may be about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight, relative to the total weight of the base lightening composition, including all ranges and subranges using any of the foregoing as upper and lower limits.

In various embodiments, it may be particularly advantageous to choose an amount of (a) ammonium persulfate that is equal to or less than the total amount of (b) additional persulfate compounds, for example less than the total amount of potassium persulfate and/or sodium persulfate. As non-limiting examples, the base lightening compositions may comprise ammonium persulfate and at least one additional persulfate compound chosen from potassium persulfate and/or sodium persulfate, where the weight ratio of (a) the amount of ammonium persulfate to (b) the total amount of additional persulfate compounds, for example potassium sulfate, or the weight ratio of the amount of ammonium persulfate to the total amount of additional persulfate compounds, for example potassium persulfate and sodium persulfate, is equal to or less than about 1. By way of example, the weight ratio may range from about 0.1 to about 0.9, such as from about 0.2 to about 0.8, from about 0.3 to about 0.7, or from about 0.4 to about 0.6. In some embodiments, the weight ratio of (a) the amount of ammonium persulfate to (b) the total amount of additional persulfate compounds, for example potassium persulfate and/or sodium persulfate, is about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, or about 0.9, or may be any range using any of the foregoing as upper and lower limits.

### Silicate Compounds

Base lightening compositions according to the disclosure comprise at least one silicate compound. Non-limiting examples of silicates include lithium, sodium, and potassium silicates, metasilicates, and disilicates, and combinations of two or more thereof, including mixed lithium, sodium, and potassium salts thereof. Specific non-limiting examples include aluminum silicate, magnesium silicate, aluminum magnesium silicate, calcium silicate, barium silicate, strontium silicate, potassium silicate, potassium metasilicate, sodium silicate, sodium metasilicate, or any combination of two or more thereof. In at least some embodiments, the base lightening compositions comprise at least one metasilicate compound, for example sodium metasilicate. In some embodiments the base lightening compositions comprise at least two silicate compounds, wherein at least one is a metasilicate.

The total amount of silicate compounds in base lightening compositions according to the disclosure may, in at least some embodiments, be greater than found in typical base lightening compositions. In particular, it may be advantageous in some embodiments to increase the amount of silicate compounds in base lightening compositions according to the disclosure to achieve and/or maintain a desired pH of the hair lightening compositions.

For example, the total amount of silicate compounds may range from about 5% to about 50%, such as from about 15% to about 45%, or from about 30% to about 40% by weight, based on the total weight of the base lightening composition. In various embodiments, the total amount of silicate compounds may range from about 5% to about 45%, about 5% to about 40%, about 5% to about 35%, about 5% to about 20%, about 10% to about 50%, about 10% to about 45%, about 10% to about 40%, about 10% to about 35%, about 10% to about 30%, about 15% to about 50%, about 15% to about 45%, about 15% to about 40%, about 15% to about 35%, about 15% to about 30%, about 20% to about 50%, about 20% to about 45%, about 20% to about 40%, about 20% to about 35%, about 20% to about 30%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, based on the total weight of the base lightening composition.

### Alkalizing Agents

The base lightening compositions according to the disclosure may optionally further comprise at least one alkalizing agent other than silicates. The alkalizing agents may be chosen from organic and/or mineral alkalizing agents, for example, organic amines and their organic or mineral salts, ammonium salts, and mineral alkalizing agents such as inorganic bases and hydroxide base compounds.

In various embodiments, it may be advantageous to include amounts of alkalizing agents in the base lightening compositions to achieve and/or maintain a desired pH of the hair lightening compositions.

### Organic Alkalizing Agents

As non-limiting examples, suitable organic amines may be chosen from alkanolamines. In various embodiment, alkanolamines may be chosen from mono-, di- or trialkylamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof. In some embodiments, alkanolamine type that may be mentioned include but not limited to: monoethanolamine (also known as ethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane.

In one embodiment, the at least one alkalizing agent comprises, consists essentially of, or consists of alkanolamines such as monoethanolamine. In one embodiment, the alkalizing agent excludes alkanolamines, but may include derivatives of alkanolamines.

Another example of organic amines that can be used include compounds of formula (I):

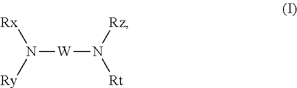

wherein:
   W is chosen from C1-C6 alkylene residues optionally substituted with a hydroxyl group or a C1-C6 alkyl radical; and
   Rx, Ry, Rz, and Rt, which may be identical or different, are chosen from a hydrogen atom, C1-C6 alkyl radicals, C1-C6 hydroxyalkyl radicals, and C1-C6 aminoalkyl radicals.

As non-limiting examples, compounds of formula (I) may be chosen from 1,3-diaminopropane, 1,3-diamino-2-propanol, spermine, and spermidine.

Useful organic salts include, by way of example only, citrates, lactates, glycolates, gluconates, acetates, propionates, fumarates, oxalates, and tartrates. Useful mineral salts include, by way of example only, hydrohalides (for example hydrochlorides), carbonates, hydrogen carbonates, sulfates, hydrogen phosphates, and phosphates.

Non-limiting examples of ammonium salts that may be used include carbonate and bicarbonate.

If present, the total amount of organic alkalizing agents may independently range from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4% by weight, based on the total weight of the base lightening composition.

### Mineral Alkalizing Agents

Useful and non-limiting mineral alkalizing agents include metal hydroxides and carbonates. Suitable metal hydroxides and carbonates include alkali metal and alkaline earth metal hydroxides or carbonates. Examples of such metal hydroxides include sodium, potassium, lithium, calcium, magnesium. In some embodiments, the alkalizing agent comprises ammonia, ammonium carbonates, sodium carbonates, potassium carbonates, ammonium bicarbonates, sodium bicarbonates, potassium bicarbonates, ammonium hydroxides, sodium hydroxides, potassium hydroxides, salts thereof, hydrates thereof, or mixtures thereof. In some embodiments, potassium carbonate may be used.

If present, the total amount of mineral alkalizing agents may range from about 0.1% to about 10% by weight, relative to the total weight of the base lightening composition, including all subranges therebetween. For example, the base lightening composition may contain from about 0.5% to about 10%, from about 0.75% to about 9%, from about 1% to about 8.5%, from about 1.5% to about 8%, from about 1.75% to about 7.5%, from about 2% to about 7%, from about 2.25% to about 6.5%, from about 2.5% to about 6%, from about 2.75% to about 5.5%, from about 3% to about 5.25%, or from about 3% to about 5% by weight, based on the total weight of the base lightening composition, including all ranges using any of the foregoing as upper and lower limits.

### Thermal Control Agents

It is known that when a base lightening composition is mixed with an aqueous developer to form a hair lightening composition, a chemical reaction occurs that raises the temperature of the mixture, for example to temperatures greater than about 70° C. (i.e. greater than about 160° F.), which can result in conditions that are uncomfortable for the user and also potentially unsafe. Therefore, in various embodiments the base lightening composition may optionally include one or more thermal control agents, for example sodium stearate, urea, mineral oil, magnesium carbonate, and/or magnesium carbonate hydroxide.

If present, the total amount of thermal control agents may vary, but typically ranges from about 0.1% to about 20%, such as from about 3% to about 18%, from about 5% to about 15%, or from about 8% to about 12%, or may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

For example, the total amount of urea may range from about 0.1% to about 15%, such as from about 0.5% to about 10%, from about 0.5% to about 8%, from about 0.5% to about 6%, from about 0.5% to about 5%, from about 0.5% to about 4%, from about 0.5% to about 3%, from about 0.5% to about 2%, from about 1% to about 10%, from about 1% to about 8%, from about 1% to about 6%, from about 1% to about 5%, from about 1% to about 4%, from about 1% to about 3%, from about 1% to about 2%, from about 1.5% to about 15%, from about 1.5% to about 10%, from about 1.5% to about 8%, from about 1.5% to about 6%, from about 1.5% to about 5%, from about 1.5% to about 4%, from about 1.5% to about 3%, from about 2% to about 10%, from about 2% to about 8%, from about 2% to about 6%, from about 2% to about 5%, from about 2% to about 4%, from about 2% to about 3%, from about 2.5% to about 10%, from about 2.5% to about 8%, from about 2.5% to about 6%, from about 2.5% to about 5%, from about 2.5% to about 4%, from about 2.5% to about 3%, from about 3% to about 10%, from about 3% to about 8%, from about 3% to about 6%, from about 3.5% to about 5%, from about 3.5% to about 4%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

As a further example, the base lightening compositions may comprise from about 0.1% to about 10%, such as from about 0.5% to about 7.5%, from about 1% to about 5%, or from about 1.5% to about 3.5% sodium stearate by weight, relative to the total weight of the base lightening composition. In some embodiments sodium stearate and mineral oil can be used together to work synergistically to control the temperature of the lightening composition without negatively impacting the level of lightening achieved. This surprising effect is most prominently seen when the weight ratio of sodium stearate to mineral oil ranges from about 1:3 to about 3:1, such as from about 1:2.5 to about 2.5:1, from about 1:2 to about 2:1, from about 1:1.5 to about 1.5:1, from about 1:1.25 to about 1.25:1, or from about 1:1.1 to about 1.1:1, or is about 1:1. If present, the amounts of sodium stearate and mineral oil may vary, but may, for example, independently range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% by weight, relative to the total weight of the base lightening composition, including all ranges and subranges using any of the foregoing as upper and lower limits.

### Oxidizing Agents

Base lightening compositions according to the disclosure optionally comprise at least one oxidizing agent other than persulfates. Useful and non-limiting examples of oxidizing agents include perborates, percarbonates, peroxides, organic peracids, salts thereof, or mixtures thereof. In some embodiments, the base lightening compositions can include, as oxidizing agents, one or more of alkali metal bromates, ferricyanides, redox enzymes such as laccases, peroxidases, solid hydrogen peroxides such as urea hydrogen peroxide, sodium peroxide, and strontium peroxide, and 2-electron oxidoreductases, such as uricase.

If present, the total amount of oxidizing agents other than persulfates in the base lightening compositions may, in various embodiments, range from about 0.5% to about 45%, such as from about 1% to about 40%, from about 1.5% to about 35%, from about 2% to about 30%, from about 2.5% to about 25%, from about 3% to about 20%, from about 3.5% to about 15%, from about 4% to about 12%, from about 4.5% to about 10%, or from about 5% to about 8% by weight, based on the total weight of the hair base lightening composition, including all ranges using any of the foregoing as upper and lower limits. In at least some embodiments, the base lightening composition is free or substantially free of oxidizing agents other than persulfates.

### Organic Acids and Salts of Organic Acids

Optionally, base lightening compositions according to the disclosure comprise at least one organic acid, for example chosen from carboxylic acids. It is believed that the use of organic acids provides advantageous bonding benefits to the hair. Salts of organic acids may also be chosen, and are expressly included in the terms “organic acid” and “carboxylic acid” whether or not so stated, unless expressly indicated otherwise.

In various embodiments, the organic acids may be chosen from non-polymeric mono-, di-, and/or tri-carboxylic acids. Non-limiting examples of useful carboxylic acids include citric acid, maleic acid, succinic acid, aspartic acid, glutamic acid, lactic acid, malic acid, tartaric acid, glutaric acid, acetic acid, glycolic acid, oxalic acid, salts thereof, or combinations thereof. Derivatives of the foregoing, e.g. keto or oxo derivatives, may also be chosen. In some embodiments, the organic acid chosen comprises, consists essentially of, or consists of citric acid and salts thereof, such as sodium citrate. In at least some preferred embodiments, sodium citrate is used.

If present, the total amount of organic acids and salts thereof may range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, or about 7% by weight, relative to the total weight of the base lightening composition, including all ranges and subranges using any of the foregoing as upper and lower limits.

### Amino Acids

Optionally, the base lightening compositions comprise one or more amino acids. It is believed that the use of amino acids provides advantageous bonding benefits to the hair, particularly in combination with organic acids and salts. Salts of amino acids may also be chosen, and are expressly included in the term “amino acid” whether or not so stated. As used herein, the term “amino acid” includes amino carboxylic acids and salts thereof as well as amino sulfonic acids and salts thereof. As used herein, amino acids are understood to refer to organic compounds containing a carboxylic acid group (—COOH) (amino carboxylic acids) and/or sulfonic acid group (—S(═O)₂—OH) (amino sulfonic acids) and an amino group (—NH₂) which may be primary or secondary, or may be intra-cyclic, along with a side chain (R group) specific to each amino acid.

Non-limiting examples of amino carboxylic acids that may be chosen include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and combinations of two or more thereof. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoluene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and combinations of two or more thereof.

By way of example, in some embodiments the base lightening compositions comprise one or more amino carboxylic acids of formula (II):

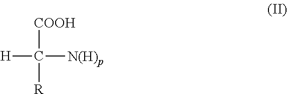

wherein:
   p is an integer equal to 1 or 2, and
   when p=1, R forms, together with the nitrogen atom, a saturated heterocycle comprising from 5 to 8 ring members, preferably 5 ring members, it being possible for this ring to be substituted by one or more groups chosen from hydroxyl or (C1-C4)alkyl; or
   when p=2, R represents a hydrogen atom or a saturated, linear, or branched (C1-C12)alkyl group, preferably a (C1-C4)alkyl group, optionally interrupted by one or more heteroatoms or groups chosen from —S—, —NH—, or —C(NH)—, and/or optionally substituted by one or more groups chosen from hydroxyl (—OH), amino (—NH2), —SH, —COOH, —CONH2, —NH—C(NH)—NH2, or an imidazole ring.

In some embodiments, the base lightening compositions comprise one or more amino acids chosen from arginine, glycine, proline, methionine, serine, lysine, histidine, salts of any of the foregoing (e.g. alkali metal, alkaline earth metal, or zinc salts), or combinations of two or more thereof. In at least certain embodiments, the amino acid compounds in the base lightening composition consist essentially of or consist of amino acids chosen from glycine or salts thereof.

In various embodiments, the total amount of amino acids may range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% by weight, relative to the total weight of the base lightening composition, including all ranges and subranges using any of the foregoing as upper and lower limits.

### Oils

In some cases, the base lightening compositions include one or more oils. Non-limiting examples of oils include ester oils, silicone oils, fluoro oils, vegetable oils, animal oils, hydrocarbon-based oils, or mixtures thereof. For example, lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, shea butter, or combinations of two or more thereof can be used. Mention is made, as an example of linear or branched hydrocarbons, of oils of mineral or synthetic origin, such as liquid paraffins and their derivatives, petrolatum, mineral oils, (mixtures of oil-derived hydrocarbon oils), polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane, and their mixtures.

The total amount of oils can vary, but typically ranges from about 0.01% to about 10%, such as from about 0.1% to about 8%, or from about 0.5% to about 6% by weight, relative to the total weight of the base lightening composition. For example, in some embodiments, the total amount of oils may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the base lightening composition.

### Fillers

Optionally, base lightening compositions according to the disclosure comprise at least one filler (other than silicates). Non-limiting examples of fillers include starches, maltodextrins, perlites, zeolites, polylactic acids, silicas, polyamide powders, polyvinylpyrrolidones, dextrose, oligosaccharides, celluloses, diatomite, diatomaceous earth, talc, clays, silicon dioxide, clays, or mixtures thereof. In some instances, the one or more fillers comprises one or more alkali metal salts of fatty acids and/or organic base salts of fatty acids, for example, sodium stearate, zinc laurate, magnesium stearate, magnesium myristate, zinc stearate, ammonium stearate, ammonium oleate, ammonium nonanoate, sodium palmitate, potassium stearate, potassium palmitate, sodium myristate, aluminum monostearate, or combinations of two or more thereof.

If present, the total amount of fillers may vary, but typically ranges from about 1% to about 20%, including all subranges therebetween, such as from about 1% to about 18%, from about 2% to about 16%, from about 2.5% to about 15%, or from about 3% to about 14%, or from about 4% to about 13% by weight, relative to the total weight of the base lightening composition, for example from about 1% to about 18%, from about 1% to about 16%, from about 1% to about 15%, from about 1% to about 14%, from about 1% to about 13%, from about 1% to about 12%, from about 1.5% to about 20%, from about 1.5% to about 16%, from about 1.5% to about 15%, from about 1.5% to about 14%, from about 1.5% to about 13%, from about 1.5% to about 12%, from about 2% to about 20%, from about 2% to about 18%, from about 2% to about 15%, from about 2% to about 14%, from about 2% to about 13%, from about 2% to about 12%, from about 2.5% to about 20%, from about 2.5% to about 18%, from about 2.5% to about 16%, from about 2.5% to about 14%, from about 2.5% to about 13%, from about 2.5% to about 12%, from about 3% to about 20%, from about 3% to about 18%, from about 3% to about 16%, from about 3% to about 15%, from about 3% to about 13%, from about 3% to about 12%, from about 4% to about 20%, from about 4% to about 18%, from about 4% to about 16%, from about 4% to about 15%, from about 4% to about 14%, or from about 4% to about 12% by weight, relative to the total weight of the base lightening composition.

### Thickening Agents

The base lightening compositions can also optionally include one or more thickening agents. Non-limiting examples of thickening agents include carboxylic acid polymers, crosslinked polyacrylate polymers, polyacrylamide polymers, polysaccharides, and gums. More specific, non-limiting examples of thickening agents include acacia, agar, algin, alginic acid, ammonium alginate, amylopectin, calcium alginate, calcium carrageenan, carnitine, carrageenan, celluloses and modified celluloses such as hydroxyethylcellulose, dextrin, gelatin, gellan gum, guar gum, guar hydroxypropyltrimonium chloride, hydroxylpropyl guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, or mixtures thereof. In some cases, the one or more thickening agents include a crosslinked polyacrylate polymer, for example, acrylates/C10-30 alkylacrylate crosspolymer. Furthermore, in some cases the one or more thickening agents include guar gum.

If present, the total amount of thickening agents may range from about 0.01% to about 10%, such as from about 0.01% to about 5%, from about 0.1% to about 5%, from about 0.5% to about 5%, from about 0.5% to about 3%, from about 1% to about 5%, from about 1% to about 3%, from about 1.25% to about 2.75%, or from about 1.5% to about 2.5% by weight, relative to the total weight of the base lightening composition.

### Anionic Surfactants

Optionally, base lightening compositions comprise one or more anionic surfactants. For instance, the anionic surfactant(s) that may be useful include alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

Use is also made of (C₆-C₂₄)alkyl sulfates, (C₆-C₂₄)alkyl ether sulfates, which are optionally ethoxylated, comprising from 2 to 50 ethylene oxide units, or mixtures thereof, in particular in the form of alkali metal salts or alkaline-earth metal salts, ammonium salts or amino alcohol salts.

In some cases, the one or more anionic surfactant is selected from the group consisting of sodium oleyl succinate, ammonium lauryl sulphosuccinate, sodium lauryl sulfate, sodium lauryl ether sulfate (also known as sodium laureth sulfate, SLES), sodium lauryl ether sulphosuccinate, ammonium lauryl sulfate (ALS), ammonium lauryl ether sulfate (ammonium laureth sulfate), sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate, or mixtures thereof. In some instances, preferred anionic surfactants include sodium lauryl sulfate, sodium lauryl ether sulfate, sodium lauryl ether sulphosuccinate, ammonium lauryl sulfate, ammonium lauryl ether sulfate, or mixtures thereof.

If present, the total amount of anionic surfactants may range from about 0.1% up to about 15%, such as up to about 12%, up to about 10%, up to about 9%, up to about 8%, up to about 7%, or up to about 6% by weight, relative to the total weight of the base lightening composition. For example, the total amount of anionic surfactants may range from about 0.1% to about 10%, from about 0.5% to about 9%, from about 1% to about 8%, or from about 2% to about 7% by weight, relative to the total weight of the base lightening composition. In at least some embodiments, the base lightening compositions comprise at least one anionic surfactant, and have a total amount of anionic surfactants ranging from about 0.5% to about 12%, such as from about 1% to about 10%, from about 1.5% to about 9%, or from about 2% to about 8%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the base lightening composition, including any range using any of the foregoing as upper and lower limits.

The base lightening composition may comprise additional auxiliary components, for example preservatives, hair conditioning agents, vitamins, chelating agents, pigments or colorants to color the composition, or any other component typically used in hair color lightening base compositions.

In some embodiments the base lightening composition will be anhydrous or substantially anhydrous, for example with less than about 1% water, such as less than about 0.5% water, or less than about 0.1% water. The form of the base lightening composition may be a powder, cream, gel, or the like.

### Base Lightening Mixtures

As discussed, the base lightening compositions use less persulfates than traditional base lightening compositions, which typically comprise at least 50% or more persulfates. However, commercially-available developer compositions are formulated to be mixed in predetermined mix ratios with traditional base lightening compositions. Therefore, in some embodiments the base lightening compositions according to the disclosure can be mixed with an appropriate amount of a solvent, such as water, to form a base lightening mixture. The base lightening mixture can then be mixed with an oxidizing composition or oxidizing agent in a desired mix ratio.

The choice of solvent is not limited other than that it will generally not significantly reduce the lightening effects, slow the lightening process, or otherwise interfere with the intended result of the process. Unexpectedly, it has been discovered that by using a solvent that comprises, consists essentially of, or consists of water a greater increase in lift can be achieved. This finding was particularly surprising as adding water would have been expected to dilute or diminish the bleaching power. As such, it may be particularly advantageous to use water as the solvent. Therefore, in various embodiments, the solvent comprises, consists essentially of, or consists of water.

As another example, the solvent can be an aqueous oxidizing (developer) composition. The choice of aqueous oxidizing composition as a solvent to prepare a base lightening mixture is not limited, and can, for example, include any of the aqueous oxidizing compositions described below. In addition, in various embodiments it may be advantageous to choose a type and/or strength of developer composition as solvent which is the same or different than that used to prepare the hair lightening composition. For example, the developer chosen as a solvent for preparing the base lightening mixture may be a 10V, 20V, 30V, or 40V aqueous hydrogen peroxide composition, which may or may not be the same as the oxidizing composition used to prepare the hair lightening composition.

The amount of solvent to be mixed with or added to the base lightening composition to prepare the base lightening mixture can be determined by the amount of persulfates and alkalizers present. Typically, traditional base lightening compositions will comprise in the range of 50-60% persulfates. Thus, for example, a base lightening composition comprising 35% persulfates and an additional 5% of alkalizers could be mixed with an amount of solvent in the range of 10-20% by weight, relative to the total weight of the base lightening composition, as a superaddition.

In various embodiments, the amount of solvent that may be mixed with or added to the base lightening composition can range from about 1% to about 25%, such as, for example, from about 1% to about 20%, from about 1.5% to about 18%, from about 2% to about 15%, from about 2.5% to about 12%, from about 3% to about 10%, from about 3.5% to about 8%, from about 4% to about 6%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 23.5%, about 24%, about 24.5%, or about 25% by weight, relative to the total weight of the base lightening composition, including all ranges and subranges using any of the foregoing as upper and lower limits.

In other embodiments, the amount of solvent added to or mixed with the base lightening composition may be chosen relative to the total amount of persulfates in the base lightening composition. For example, the weight ratio of total persulfates in the base lightening composition to the amount of solvent may range from about 1:1 to about 10:1, such as from about 1:1 to about 5:1, or from about 1.5:1 to about 3.5:1. In various embodiments, the weight ratio of the total amount of persulfates in the base lightening composition to the amount of solvent added to or mixed with the base lightening composition may be about 1:1, about 1.25:1, about 1.5:1, about 1.75:1, about 2:1, about 2.25:1, about 2.5:1, about 2.75:1, about 3:1, about 3.25:1, about 3.5:1, about 3.75:1, about 4:1, about 4.25:1, about 4.5:1, about 4.75:1, or about 5:1, or may be any range using any of the foregoing as upper and lower limits.

In other embodiments still, the amount of solvent added to or mixed with the base lightening composition may be chosen relative to the total weight of the base lightening composition. For example, the weight ratio of total weight of the base lightening composition to solvent may range from about 5:1 to about 15:1, such as from about 6:1 to about 14:1, or from about 7:1 to about 13.5:1. In various embodiments, the weight ratio of the total weight of the base lightening composition to the amount of solvent added to or mixed with the base lightening composition may be about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, about 10:1, about 10.25:1, about 10.5:1, about 10.75:1, about 11:1, about 11.25:1, about 11.5:1, about 11.75:1, about 12:1, about 12.25:1, about 12.5:1, about 12.75:1, about 13:1, about 13.25:1, about 13.5:1, about 13.75:1, about 14:1, about 14.25:1, about 14.5:1, about 14.75:1, or about 15:1, or may be any range using any of the foregoing as upper and lower limits.

Although not necessary, it may be advantageous for the user to combine the base lightening composition and solvent to prepare the base lightening mixture at or near the time of use, for example just before or simultaneously with mixing the base lightening composition and oxidizing composition/oxidizing agent. It should be understood that the order of mixing is not critical. Alternatively, the solvent may be added to the base lightening composition during manufacture.

### Oxidizing Compositions and Oxidizing Agents

The oxidizing composition (developer) or oxidizing agent that can be mixed with the base lightening composition or mixture to prepare the hair lightening composition at or near the time of use is not limited, and any conventional oxidizing composition or oxidizing agent for lightening hair can be used. Typically an oxidizing composition comprises an oxidizing agent such as peroxide, inorganic perhydrate salts, or a source of peroxymonocarbonate ions, in a cosmetically acceptable carrier. Non-limiting examples of peroxides include hydrogen peroxide, inorganic alkali metal peroxides such as sodium peroxide, and organic peroxides such as urea peroxide and melamine peroxide. Non-limiting examples of inorganic perhydrate salts include the alkali metal salts of perborates, percarbonates, perphosphates, persilicates, persulphates, and the like. These inorganic perhydrate salts may optionally be used as monohydrates, tetrahydrates, etc.

As non-limiting examples of sources of peroxymonocarbonate ions, in situ formation from a source of hydrogen peroxide and a source of hydrogen carbonate ions can be used. This system is particularly effective in combination with a source of ammonia or ammonium ions. Accordingly, any source of peroxymonocarbonate ions may be utilized. Suitable sources include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, barium carbonate, ammonium carbonate, ammonium hydrogen carbonate, and mixtures thereof. Percarbonate salts may also be utilized to provide both the source of carbonate ions and as an oxidizing agent. Non-limiting examples of carbonate ions, carbamate, and hydrocarbonate ions are sodium hydrogen carbonate, potassium hydrogen carbonate, ammonium carbamate, and mixtures of two or more thereof.

The amount of oxidizing agent will vary, but typically ranges from about 1% to about 40%, such as from about 2% to about 30%, from about 2% to about 25%, from about 2% to about 20%, from about 2% to about 15%, from about 2% to about 10%, from about 2% to about 5%, from about 5% to about 30%, from about 5% to about 25%, from about 5% to about 20%, from about 5% to about 15%, from about 5% to about 10%, from about 10% to about 30%, from about 10% to about 25%, from about 10% to about 20%, or from about 10% to about 15% by weight, relative to the total weight of the oxidizing composition, including any range using any of the foregoing as upper and lower limits. By way of example only, the oxidizing agent may comprise, consist essentially of, or consist of hydrogen peroxide, present in any of the foregoing ranges, for example about 3%, about 6%, about 12%, or about 18% by weight, relative to the total weight of the oxidizing composition. However, because oxidizing agents also damage the hair, it would be advantageous to reduce the amount needed to achieve the same or even greater levels of lightening.

The oxidizing composition may further comprise additional components such as thermal control agents as described above, oils as described above, anionic surfactants as described above, thickening agents as described above, organic acids and their salts as described above, amino acids and their salts as described above, nonionic surfactants, cationic surfactants, amphoteric surfactants, hair conditioning agents, preservatives, vitamins, chelating agents, pigments or colorants to color the composition, pH adjusters, fragrances, or any other component typically used in developer compositions for hair lightening.

The oxidizing composition may be in the form of a powder, gel, liquid, foam, lotion, cream, mousse, or emulsion. In some embodiments, the oxidizing composition is anhydrous, and is mixed with water prior to or simultaneously with being mixed with a base lightening composition or mixture. The oxidizing composition may optionally be aqueous, and have a pH less than 7, such as ranging from about 1 to about 6 or about 2 about 5. For example, the oxidizing composition may be a 10V, 20V, 30V, or 40V aqueous hydrogen peroxide composition, and have a pH ranging from about 2.5 to about 4.5.

### Hair Lightening Compositions

Hair lightening compositions comprise the components of base lightening compositions according to the disclosure, optionally one or more solvents that is (are) mixed with the base lightening composition, and an oxidizing agent. Typically, a base lightening composition and an oxidizing composition (developer) are mixed at or near the time of use in a predetermined mix ratio, depending on strength and level of lift to be achieved. Such predetermined ratios typically range from about 1:3 to about 3:1, such as from about 1:2.5 to about 2.5:1, from about 1:2 to about 2:1, or from about 1:1.5 to about 1.5:1, for example about 1:1.

In embodiments where one or more solvents is mixed with the base lightening composition to form a base lightening mixture, the base lightening mixture and oxidizing composition comprising the oxidizing agent may be mixed at or near the time of use, for example in a mix ratio of [base lightening mixture]:[oxidizing composition] ranging from about 1:3 to about 3:1, such as from about 1:2.5 to about 2.5:1, from about 1:2 to about 2:1, or from about 1:1.5 to about 1.5:1, such as about 1:1, to form the hair lightening composition. For example, the mix ratio of [base lightening mixture]:[oxidizing composition] may be about 1:1, about 1:1.5, or about 1:2.

In embodiments where an additional amount of oxidizing composition is used as a solvent, a base lightening mixture may, in some cases, not be prepared prior to forming the hair lightening composition; rather, the base lightening composition may be mixed directly with the oxidizing composition. In such embodiments, a base lightening composition is mixed with the oxidizing composition in an “effective mix ratio” that can be determined by adding the amount of base lightening composition and the amount of additional oxidizing composition used, and subtracting the amount of additional oxidizing composition from the total amount of oxidizing composition. Thus, an “effective mix ratio” can be calculated as [base lightening composition+additional oxidizing composition]:[total oxidizing composition−additional oxidizing composition]. Exemplary effective mix ratios may range from about 1:3 to about 3:1, such as from about 1:2.5 to about 2.5:1, from about 1:2 to about 2:1, or from about 1:1.5 to about 1.5:1, such as about 1:1, to form the hair lightening composition. For example, the effective mix ratio of [base lightening composition+additional oxidizing composition]:[total oxidizing composition−additional oxidizing composition] may be about 1:1, about 1:1.5, or about 1:2.

As such, the amounts of components described herein for the base lightening composition and oxidizing composition will be present in the hair lightening composition in amounts consistent with the chosen mix ratio. By way of illustration only, if a 1:1 mix ratio of [base lightening mixture]:[oxidizing composition] is used, the amounts of components in the oxidizing composition will be present in the hair lightening composition at 50% of the amounts described herein. The amounts of components in the base lightening composition will likewise be present in the hair lightening composition at 50% of the amounts described herein for the base lightening composition, less the amount of solvent added.

The hair lightening composition will typically have a pH ranging from about 8 to about 13, such as from about 9 to about 12, from about 9.5 to about 11, from about 10 to about 11, or from about 10 to about 10.5.

Hair lightening compositions according to the disclosure are surprisingly effective at providing very high or even ultra-high levels of lightening in a shorter period of time and/or using lower volumes of developer compared to traditional hair lightening compositions, despite the reduced amounts of persulfates. Since the exposure of the hair to the lightening composition is reduced, the treated hair also has less damage, is stronger, and appears and feels healthier compared to hair treated with traditional hair lightening compositions.

The hair lightening compositions are stable and have a smooth, light, whip cream-like texture upon mixing the base lightening composition or base lightening mixture and developer, which texture remains during the lightening process. This is in contrast to traditional hair lightening mixtures which become thick during application and solid and crumbly during the lightening process.

Hair lightening compositions according to the disclosure effectively control the peak temperature that is attained during the lightening process. In various embodiments, the peak temperature is less than 70° C., for example less than about 68° C., less than about 65° C., less than about 63° C., less than about 60° C., less than about 58° C., less than about 55° C., less than about 53° C., or less than about 50° C.

### II. Methods

The disclosure further relates to methods of preparing hair lightening compositions and methods of lightening hair. The methods comprising mixing a base lightening composition according to the disclosure and an oxidizing composition at or near the time of use and applying the resulting mixture (hair lightening composition) to the hair.

Methods of preparing hair lightening compositions according to the disclosure may, in various embodiments, comprise one or two mixing steps. For example, a two-step method may comprise first mixing a base lightening composition with a solvent to form a base lightening mixture, and second mixing the base lightening mixture with an oxidizing composition to form the hair lightening composition. An alternative method of preparing a hair lightening composition may comprise first mixing the solvent with the oxidizing composition, and subsequently adding or mixing the base lightening composition therewith. As yet another example, methods of preparing a hair lightening composition may comprise first mixing a base lightening composition with an oxidizing composition, and subsequently adding or mixing the solvent therewith. A non-limiting example of a one-step method includes mixing a base lightening composition with an oxidizing composition, where the oxidizing composition comprises an additional amount of oxidizing composition as the solvent. Optionally, the first and/or second adding or mixing steps may be performed at or near the time of use to prepare the hair lightening composition.

In an exemplary embodiment, a method of preparing a hair lightening composition according to the disclosure comprises mixing (a) a base lightening composition comprising (i) from about 5% to about 20%, for example from about 8% to about 15%, of ammonium persulfate, (ii) from about 10% to about 40%, for example from about 15% to about 35%, of one or more additional persulfate compounds, and (iii) from about 5% to about 50% of one or more silicate compounds, (b) an amount of solvent ranging from about 1% to about 25%, for example from about 2% to about 15%, and (c) an oxidizing agent, for example an aqueous 10V, 15V, 20V, 25V, 30V, 35V, or 40V hydrogen peroxide developer, where the weight ratio of the total amount of ammonium persulfate to the total amount of additional persulfate compounds in the base lightening composition is equal to or less than about 1, for example ranges from about 0.1 to about 0.9, and where all amounts are by weight relative to the total weight of the base lightening composition. The components (a), (b), and (c) may be mixed in any order, and in a mix ratio of [(a)+(b)]:(c) ranging from about 1:3 to about 3:1, such as about 1:2 to about 2:1, about 1:1.5 to about 1.5:1, or about 1:1.

In another exemplary embodiment, a method of preparing a hair lightening composition according to the disclosure comprises mixing (a) a base lightening composition comprising (i) from about 10% to no more than 45% by weight, relative to the total weight of the base lightening composition, for example from about 15% to no more than 43%, of one or more persulfate compounds, and (ii) from about 5% to about 50% of one or more silicate compounds, (b) a solvent, and (c) an oxidizing agent, for example an aqueous 10V, 15V, 20V, 25V, 30V, 35V, or 40V hydrogen peroxide developer, where the weight ratio of the total amount of (i) persulfate compounds in the (a) base lightening composition to the total amount of (b) solvent ranges from about 1:1 to about 1:10, for example from about 1:1 to about 5:1, or from about 1.5:1 to about 3.5:1. The components (a), (b), and (c) may be mixed in any order, and in a mix ratio of [(a)+(b)]:(c) ranging from about 1:3 to about 3:1, such as about 1:2 to about 2:1, about 1:1.5 to about 1.5:1, or about 1:1.

In another embodiment, the solvent and base lightening composition may be mixed during manufacture, and the pre-mixed base lightening mixture will be provided to the consumer or colorist. Thus, the consumer or colorist will then mix the base lightening mixture and oxidizing composition at the desired mix ratio at or near the time of use to prepare the hair lightening composition.

Methods of lightening the color of hair are known. Typically, after the base lightening composition and developer composition are mixed, the hair lightening composition is applied to the hair (e.g., by brush, foam applicator, etc.), optionally the treated hair is covered, e.g., with foil, the composition allowed to remain on the hair for a time sufficient to achieve the desired alteration in the color of the hair (processing time), and then the composition is removed from the hair, e.g., by wiping, rinsing, and/or shampooing the hair.

It may, in various embodiments, be advantageous to employ a single application of the hair lightening composition to the hair, for example when using medium or higher levels of oxidizing agents such as 30V or greater aqueous hydrogen peroxide developers (or equivalent), or to apply the hair lightening composition more than one time, for example when using medium or lower levels of oxidizing agents such as 30V or lower aqueous hydrogen peroxide developers (or equivalent). For example, if a single application is contemplated, the hair lightening composition may be applied to the hair, allowed to process for a desired processing time, and then the hair may be rinsed and/or shampooed. In embodiments where more than one application is contemplated, the hair lightening composition may be applied to the hair, allowed to process for a desired processing time, all or substantially all of the composition removed from the hair, a second application of the hair lightening composition applied to the hair and the hair allowed to process for a second desired processing time, and then the hair may be rinsed and/or shampooed. The first hair lightening composition can be removed from the hair by any method, for example wiping the hair, scraping the hair, rinsing the hair, etc.

In embodiments where more than one application of hair lightening composition is contemplated, the second application of hair lightening composition is understood to occur within the same service or treatment, e.g. within the same day (24 hours or less) as the first application, such as within less than 12 hours after the first application, within less than 6 hours after the first application, within less than 4 hours after the first application, within less than 3 hours after the first application, within less than 2 hours after the first application, or within less than 1 hour after the first application.

In embodiments where more than one application of hair lightening composition is contemplated, the hair lightening composition used in a second application may be the same or different as the first hair lightening composition, and/or the second processing time may be the same or different as the first processing time. It may, for example, be possible to use a second hair lightening composition according to the disclosure that is different than the first hair lightening composition according to the disclosure, or it may be possible to use a second hair lightening composition other than those described herein. It has been surprisingly discovered that hair lightening compositions according to the disclosure provide higher levels of lift when multiple applications of a hair lightening composition are employed compared to a single application, even when the overall processing times are the same. Therefore, a multiple application method can provide higher levels of lift than a single application method, and/or reduced processing times to achieve the same level of lift as a single application method. For example, it may be possible to obtain very high or even ultra-high lift with a multiple application method as described herein with hair lightening compositions according to the disclosure that use lower volumes of developer. For example, a 20V or 25V developer may give increased ΔE and/or ΔL values with a multiple application method, while a single application method may give the same or increased ΔE and/or ΔL values when a 30V or 40V developer is used.

It should also be understood that more than two applications of hair lightening composition can be employed in multiple application methods, following the same general protocol. For example, a third application of hair lightening composition, which may be the same as or different than the first and/or second hair lightening compositions may be applied to the hair, and the hair left to process for a processing time that is the same as or different than the first and/or second processing times.

Processing times needed to achieve the desired lightening of the color of the hair, e.g., desired degree or level of lift of the color of the hair, vary. Exemplary processing times may range from about 5 minutes to about 75 minutes, such as about 10 minutes to about 70 minutes, about 15 minutes to about 65 minutes, about 20 minutes to about 60 minutes, about 25 minutes to about 55 minutes, about 30 minutes to about 50 minutes, or about 30 minutes to about 40 minutes, including all ranges and subranges using any of the aforementioned as upper and lower limits. In preferred embodiments, the processing time is less than 60 minutes, such as less than 55 minutes, for example is 50 minutes, 45 minutes, 40 minutes, 35 minutes, 30 minutes, 25 minutes, 20 minutes, 15 minutes, or 10 minutes. It should be understood that for multiple-application methods of lightening hair, each processing time can be in the above ranges. Thus, for example, a first processing time may be about 50 minutes and a second processing time may be about 40 minutes, for a total processing of about 90 minutes.

Surprisingly, the amount of time needed to achieve the same or even higher levels of lift with hair lightening compositions according to the disclosure is reduced compared to traditional hair lightening compositions. For example, the processing time to achieve the same or even higher levels of lift may be reduced by about or at least 5%, about or at least 10%, about or at least 15%, about or at least 20%, about or at least 25%, or about or at least 30%. In preferred embodiments, the processing time ranges from at least 20 minutes to less than 60 minutes.

Optionally, processing may take place at room temperature or at elevated temperatures (e.g., under a hood dryer).

Thus, in various embodiments, methods according to the disclosure comprise applying a hair lightening composition to the keratin materials to be treated, e.g. hair growing from the scalp, leaving the hair lightening composition on the hair for a processing time as described above, for example ranging from about 20 minutes to about 60 minutes, or about 30 minutes to about 50 minutes, with or without the use of heat and/or foils, and subsequently removing the hair lightening composition from the hair. It is also possible to treat the hair with the hair lightening composition more than one time, e.g. two times (double processing) or more. In such embodiments, the composition will be applied to the hair, left on the hair for a desired length of time, wiped from the hair (typically without rinsing), and the hair lightening composition applied to the hair a second time and left on the hair for a desired length of time. A further wiping/reapplication can be done, or the hair can be rinsed and dried.

In additional embodiments, the methods may comprise reducing hair damage caused by lightening the hair by using hair lightening compositions according to the disclosure, compared to the damage caused by conventional hair lightening compositions.

In some embodiments, the methods comprise an additional step of mixing a hair base lightening composition with a solvent, for example at or near the time of use, and mixing the hair base mixture with an oxidizing composition or oxidizing agent at or near the time of use. It should be understood that the solvent in such embodiments could alternatively be mixed with the oxidizing agent or composition at or near the time of use. In other embodiments the methods comprise mixing an additional amount of oxidizing composition with a base lightening composition and/or oxidizing composition at or near the time of use. It should also be understood that the order of mixing is not limited, nor is the timing of mixing provided the composition is suitable for lightening the color of the hair.

### III. Kits

The disclosure further relates to kits that can be used in methods of lightening hair. The kits comprise at least two compartments or containers, such as at least three compartments or containers or more. The first compartment or container comprises a base lightening composition according to the disclosure. The second compartment or container may comprise an oxidizing composition. Alternatively, the second compartment or container may comprise an implement to mix the base composition and an oxidizing composition, and/or to apply the mixture to the hair, for example a bowl and/or a brush.

In an exemplary embodiment, the kit comprises at least three compartments or containers, where the first compartment or container comprises a base lightening composition according to the disclosure, a second compartment or container comprises an oxidizing composition, and a third compartment or container comprises an implement to mix the base composition and an oxidizing composition, and/or to apply the mixture to the hair. In such an embodiment, the hair lightening composition prepared by mixing the contents of the first and second compartments or containers would result in a hair lightening composition according to the disclosure.

In another exemplary embodiment, the kit comprises at least three compartments or containers where the first compartment or container comprises a base lightening composition according to the disclosure, a second compartment or container comprises an oxidizing composition, and a third compartment or container comprises a solvent according to the disclosure. In such an embodiment, the hair lightening composition prepared by mixing the contents of the first, second, and third compartments or containers would result in a hair lightening composition according to the disclosure.

In yet a further exemplary embodiment, the kit comprises at least three compartments or containers, where the first compartment or container comprises a base lightening composition according to the disclosure excluding one or more of the components described herein, for example excluding thermal control agents. In such an embodiment, a second compartment or container comprises the excluded component(s), for example comprises one or more thermal control agents, and a third compartment or container comprises an oxidizing composition. In such an embodiment, the hair lightening composition prepared by mixing the contents of the first, second, and third compartments or containers would result in a hair lightening composition according to the disclosure just as if the thermal control agent(s) had been included in the base lightening composition. Optionally, a fourth compartment or container which comprises an implement to mix the base composition and an oxidizing composition, and/or to apply the mixture to the hair may be included.

In still further exemplary embodiments, the kits may comprise at least one additional compartment or container comprising a solvent to be mixed with the base lightening composition. In further exemplary embodiments still, the kits may comprise at least one additional compartment or container comprising one or more additional hair care compositions, for example comprising a pre- and/or post-treatment hair conditioning composition.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “citric acid and/or salts thereof” means “citric acid and salts thereof” as well as “citric acid or salts thereof.”

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−5% from the stated number, such as +/−5%, +/−4%, +/−3%, +/−2%, or +/−1%. Unless expressly stated otherwise, “about” means+/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions in which the component is present, unless otherwise indicated.

The term “altering the color” or “color altering” as used herein refers to lifting or lightening the color of hair.

For purposes of the present disclosure, naturally-straight, natural level 4 hair that has a ΔL value of about 58 or higher when treated with a hair lightening composition as described herein is considered to achieve “very high” levels of light or lightening, and naturally-straight, natural level 4 hair that has a ΔL value of about 62 or higher when treated with a hair lightening composition as described herein is considered to achieve “ultra-high” levels of lift or lightening, when measured in the CIE L*a*b* system using ColorShot MS (Newtone Technologies, software version 1.8.0).

As used herein, the terms “applying a composition onto keratin fibers” and “applying a composition onto hair” and variations of these phrases are intended to mean contacting the keratin fibers including hair, with at least one of the compositions of the disclosure, in any manner. It may also mean contacting the keratin fibers or hair in an effective amount of the composition.

As used herein, the term “salts” may include salts having a counter-ion such as an alkali metal, alkaline earth metal, or ammonium counterion. This list of counterions, however, is non-limiting. Salts also include a dissociated form of a compound, e.g. in an aqueous solution.

Unless otherwise specified for a particular component, as used herein the terms “substantially free” or “essentially free” mean the component may be added in amounts less than 2% by weight, based on the total weight of the composition in which it is present, such as less than 1.75%, less than 1.5%, less than 1.25%, less than 1%, less than 0.75%, less than 0.5%, less than 0.25%, less than 0.1%, less than 0.05%, or less than 0.01% of the specified component. Unless expressly stated otherwise, “substantially free” or “essentially free” means less than 2%. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein mean that the specific component is not added to the composition but may still be present as an additive in small amounts of a raw material that is included in the composition.

As used herein, the term “surfactants,” as well as any specifically identified surfactants, includes salts of the surfactants even if not explicitly stated.

As used herein, the term “treat” (and its grammatical variations) refers to the application of the compositions of the present disclosure onto the surface of the specified keratin material, such as hair growing from the scalp.

As used herein, the phrase “at or near the time of use” is meant to indicate that the step of mixing or combining components or compositions is performed within a short time before the mixture is applied to the keratin fibers to be treated. For example, the consumer or professional colorist may perform the mixing step within an hour or less of the mixture being applied to the hair, such as 45 minutes or less, 30 minutes or less, or 15 minutes or less.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts in each composition are expressed in percentage by weight (wt %) of active materials, unless otherwise defined, relative to the total weight of the composition.

In the Examples, the change in the color of hair was evaluated with the CIE L*a*b* system using Colorshot MS (Newtone Technologies, software version 1.8.0), where the change is determined by evaluating the color of the hair after treatment (L*₂, a*₂, b*₂) compared to the color of the hair before treatment (L*₁, a*₁, b*₁). In this system, L* indicates lightness, a* indicates red/green color or tone, and b* indicates blue/yellow color or tone. The change in lightness (ΔL) and blue/yellow tones (Δb) of the hair, which can be used to evaluate the level of lift achieved, is calculated with equations (X¹) and (X²), as follows:

$$Δ L = ( {L}_{2}^{⋆} ) - ( {L}_{1}^{⋆} ) \qquad ( {X}^{1} )$$

$$Δ b = ( {b}_{2}^{⋆} ) - ( {b}_{1}^{⋆} ) . \qquad ( {X}^{2} )$$

The change in color (ΔE, 1976) is calculated with equation (X³) as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} . \qquad ( {X}^{3} )$$

### Example 1—Base Lightening Compositions and Oxidizing Compositions

Exemplary base lightening compositions 1A-1E and comparative base lightening composition C1 shown in Table 1A were prepared.

TABLE 1A
Base Lightening Compositions

| | 1A | 1B | 1C | 1D | 1E | C1 |
|---|---|---|---|---|---|---|
| AMMONIUM | 12.80 | 13.35 | 12.80 | 13.03 | 11.60 | 11.60 |
| PERSULFATE | | | | | | |
| POTASSIUM | 29.35 | 24.15 | 29.35 | 23.60 | 17.00 | 41.60 |
| PERSULFATE | | | | | | |
| MINERAL | 2.21 | 2.30 | 2.21 | 2.25 | 4.60 | 2.00 |
| OIL | | | | | | |
| SODIUM | 4.17 | 4.35 | 4.17 | 4.25 | 4.60 | 3.78 |
| STEARATE | | | | | | |
| SODIUM | 24.67 | 30.20 | 24.67 | 29.55 | 32.00 | 16.72 |
| SILICATE | | | | | | |
| SODIUM | 6.22 | 6.50 | 6.22 | 6.36 | 2.00 | 5.64 |
| METASILICATE | | | | | | |
| MAGNESIUM | 9.93 | 10.35 | 9.93 | 10.11 | 4.40 | 9.00 |
| CARBONATE | | | | | | |
| HYDROXIDE | | | | | | |
| HYDROXYETHYL- | 0.77 | 0.80 | 0.77 | 0.79 | 2.6 | 0.70 |
| CELLULOSE | | | | | | |
| ACRYLATES/ | 0.77 | 0.80 | 0.77 | 0.79 | 1.8 | 0.70 |
| C10-30 | | | | | | |
| ALKYL | | | | | | |
| ACRYLATE | | | | | | |
| CROSSPOLYMER | | | | | | |
| SODIUM | 6.23 | 4.20 | 4.02 | 4.10 | 3.4 | |
| CITRATE | | | | | | |
| GLYCINE | | | 2.21 | 2.25 | 2.6 | 2.00 |
| CITRIC | | | | | | 3.64 |
| ACID | | | | | | |
| DISODIUM | 1.10 | 1.15 | 1.10 | 1.12 | 1.0 | 1.00 |
| EDTA | | | | | | |
| ULTRA- | 0.68 | 0.70 | 0.68 | 0.68 | 0.4 | 0.62 |
| MARINES + | | | | | | |
| KAOLIN | | | | | | |
| SODIUM | 1.10 | 1.15 | 1.10 | 1.12 | 1.0 | 1.00 |
| LAURYL | | | | | | |
| SULFATE | | | | | | |
| TOTAL | 42.15 | 37.50 | 42.15 | 36.63 | 28.6 | 53.20 |
| PERSULFATES | | | | | | |

Oxidizing compositions (developers) D1-D7 shown in Table 1B were used.

TABLE 1B
Oxidizing Compositions

| | D1 | D2 | D3 | D4 | D5 | D6 | D7 |
|---|---|---|---|---|---|---|---|
| TRIDECETH-2 | 0.85 | 0.85 | 0.85 | 0.85 | 0.85 | 0.85 | |
| CARBOXAMIDE | | | | | | | |
| MEA | | | | | | | |
| TETRASODIUM | 0.20 | 0.20 | 0.20 | 0.20 | 0.20 | 0.20 | 0.12 |
| ETIDRONATE | | | | | | | |
| SODIUM | 0.04 | 0.04 | 0.04 | 0.04 | 0.04 | 0.04 | 0.05 |
| SALICYLATE | | | | | | | |
| GLYCERIN | 0.50 | 0.50 | 0.50 | 0.50 | 0.50 | 0.50 | |
| CETEARYL | 2.28 | 2.28 | 2.28 | 2.28 | 2.28 | 2.28 | 3.15 |
| ALCOHOL | | | | | | | |
| CETEARETH-25 | 0.57 | 0.57 | 0.57 | 0.57 | 0.57 | 0.57 | |
| HYDROGEN | 12.00 | 9.00 | 7.50 | 6.00 | 4.50 | 3.00 | 9.00 |
| PEROXIDE | | | | | | | |
| TETRASODIUM | 0.04 | 0.04 | 0.04 | 0.04 | 0.04 | 0.04 | 0.05 |
| PYROPHOSPHATE | | | | | | | |
| PEG-40 | | | | | | | 0.90 |
| HYDROGENATED | | | | | | | |
| CASTOR OIL | | | | | | | |
| SODIUM | | | | | | | 0.45 |
| CETEARYL | | | | | | | |
| SULFATE | | | | | | | |
| MINERAL | | | | | | | 17.00 |
| OIL | | | | | | | |
| WATER | QS | QS | QS | QS | QS | QS | QS |

### Example 2—Hair Lightening Compositions

Just before use, 89 grams of base lightening composition 1A was mixed with 11 grams of water to form base lightening mixture 1A′ according to the disclosure. Base lightening mixture 1A′ was then mixed with 150 grams of one of developer compositions D1-D6 to produce hair lightening compositions 2A-1 through 2A-6 according to the disclosure, as shown in Table 2A.

TABLE 2A
Exemplary Hair Lightening Compositions: Base + Solvent

| Hair Lightening | Base Lightening | | |
|---|---|---|---|
| Composition | Mixture | Developer | Mix Ratio |
| 2A-1 | 1A′ | D1 | 1:1.5 |
| 2A-2 | | D2 | 1:1.5 |
| 2A-3 | | D3 | 1:1.5 |
| 2A-4 | | D4 | 1:1.5 |
| 2A-5 | | D5 | 1:1.5 |
| 2A-6 | | D6 | 1:1.5 |

Just before use, 100 grams of base lightening composition 1A was mixed with 150 grams of one of developer compositions D1-D6, to produce hair lightening compositions 2B-1 through 2B-6 according to the disclosure, as shown in Table 2B.

TABLE 2B
Exemplary Hair Lightening Compositions

| Hair Lightening | Base Lightening | | |
|---|---|---|---|
| Composition | Composition | Developer | Mix Ratio |
| 2B-1 | 1A | D1 | 1:1.5 |
| 2B-2 | | D2 | 1:1.5 |
| 2B-3 | | D3 | 1:1.5 |
| 2B-4 | | D4 | 1:1.5 |
| 2B-5 | | D5 | 1:1.5 |
| 2B-6 | | D6 | 1:1.5 |

Just before use, comparative base lightening composition C1 was mixed with one of developer compositions D1-D7 to produce hair comparative hair lightening compositions 2C-1 through 2C-7, shown in Table 2C.

TABLE 2C
Comparative Hair Lightening Compositions

| Hair Lightening | Base Lightening | | |
|---|---|---|---|
| Composition | Composition | Developer | Mix Ratio |
| 2C-1 | C1 | D1 | 1:1.5 |
| 2C-2 | | D2 | 1:1.5 |
| 2C-3 | | D3 | 1:1.5 |
| 2C-4 | | D4 | 1:1.5 |
| 2C-5 | | D5 | 1:1.5 |
| 2C-6 | | D6 | 1:1.5 |
| 2C-7 | | D7 | 1:1.5 |

Just before use, 87 grams of base lightening composition 1B was mixed with 13 grams of water to form base lightening mixture 1B′, 91 grams of base lightening composition 1C was mixed with 9 grams of water to form base lightening mixture 1C′, and 89 grams of base lightening composition 1D was mixed with 11 grams of water to form base lightening mixture 1D. Base lightening mixtures 1B′-1C′ were then mixed with one of developer compositions D1, D2, or D4 to produce hair lightening compositions 2D-1 through 2F-3, as shown in Table 2D.

TABLE 2D
Exemplary Hair Lightening Compositions: Base + Solvent

| Hair Lightening | Base Lightening | | |
|---|---|---|---|
| Composition | Mixture | Developer | Mix Ratio |
| 2D-1 | 1B′ | D1 | 1:1.5 |
| 2D-2 | | D2 | 1:1.5 |
| 2D-3 | | D4 | 1:1.5 |
| 2E-1 | 1C′ | D1 | 1:1.5 |
| 2E-2 | | D2 | 1:1.5 |
| 2E-3 | | D4 | 1:1.5 |
| 2F-1 | 1D′ | D1 | 1:1.5 |
| 2F-2 | | D2 | 1:1.5 |
| 2F-3 | | D4 | 1:1.5 |

Hair lightening composition 2G-1 was prepared by mixing 83 grams of base lightening composition 1E with developer D1 at an effective mix ratio of 1:2. The effective mix ratio is essentially the same as found in Table 2A [83 grams base lightening composition+17 grams additional developer]:[200 grams of developer]. In this example, additional aqueous oxidizing composition (developer) took the place of the water additive and showed that it was just as effective.

### Example 3—Evaluation of Lightening Benefits

In order to evaluate the lightening efficacy of hair lightening compositions having reduced amounts of persulfates according to the disclosure compared to traditional hair lightening compositions under various conditions, the following studies were performed.

### Example 3A

Each of the hair lightening compositions in Tables 2A-2C was applied to separate swatches of natural level 4 hair (18 swatches total) at a rate of about 10 grams per gram of hair and covered with foil. The swatches were left to process at 33° C. for 50 minutes, rinsed, shampooed with a commercial shampoo, rinsed again, and blow dried. The change in lightness (ΔL) of the treated hair was then evaluated using Colorshot MS, as described above.

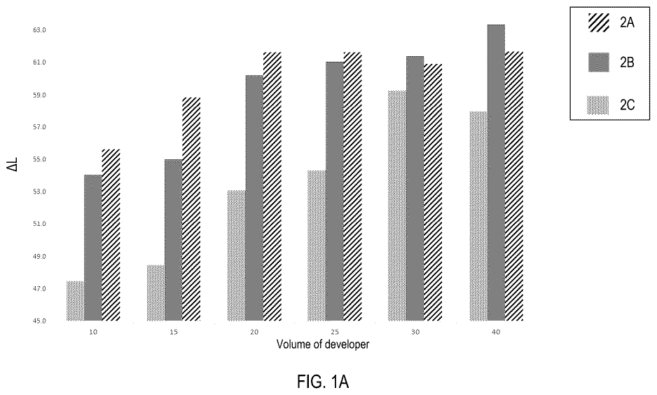

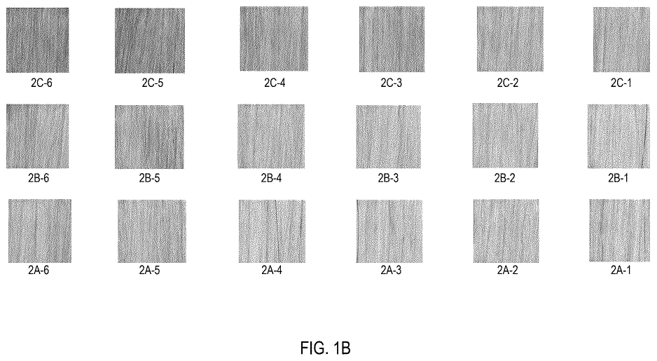

The results are shown in FIGS. 1A-1B. As seen, hair treated with hair lightening compositions 2A-1 through 2A-6 and 2B-1 through 2B-6 according to the disclosure demonstrated significantly greater lightening benefits than hair treated with comparative hair lightening compositions 2C-1 through 2C-6. In FIG. 1A, it can be seen that at every strength (volume) of developer, when processed for the same amount of time under the same conditions, hair lightening compositions according to the disclosure (2A, 2B) provided significantly increased levels of lightening (ΔL) compared to the traditional hair lightening composition (2C). These results are shown visually in FIG. 1B.

These results demonstrate that reducing the amount of harsh persulfate compounds and increasing the amount of solvent surprisingly results in greater lightening effect over the same period of time, at all of the different strengths of oxidizing agents evaluated.

### Example 3B

A study was performed where each of hair lightening compositions 2A-1, 2C-1, 2D-1, and 2D-3 was applied to five sets of swatches of natural level 4 hair (20 swatches total) at a rate of about 10 grams per gram of hair and covered with foil. The swatches were left to process at 33° C. for 15, 25, 35, 40, or 50 minutes. Once the specified processing time for each swatch ended, the swatch was rinsed, shampooed with a commercial shampoo, rinsed again, and blow dried. The change in lightness (ΔL) of the treated hair was then evaluated using Colorshot MS, as described above.

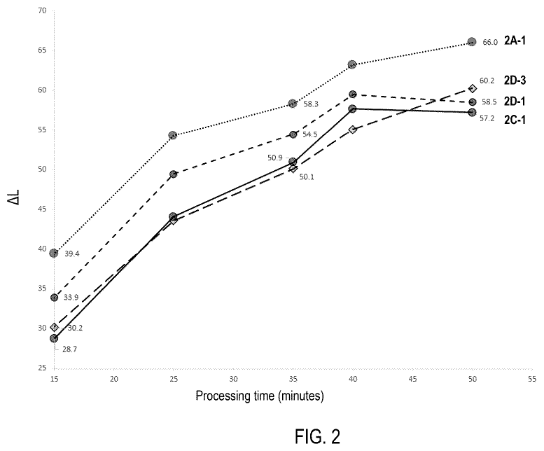

The results are shown in FIG. 2. As can be seen, when treated hair was processed for the same amount of time, hair lightening compositions 2A-1 and 2D-1 according to the disclosure having the same strength (volume) of developer as the traditional hair lightening composition (2C-1) provided significantly increased levels of lightening (ΔL) at almost every time point. While hair lightening composition 2D-3 with half the strength (volume) of developer performed better in terms of lightening at 15 minutes than the traditional hair lightening composition (2C-1), FIG. 2 shows that in the range of 25-35 minutes the level of lightening was approximately the same, and from 35-40 minutes composition 2C-1 had greater lightening, but after 40 minutes the lightening of composition 2C-1 leveled out whereas composition 2D-3 continued to increase in lightening effects, ultimately outperforming composition 2C-1.

These results surprisingly demonstrate that reducing the amount of persulfate compounds and increasing the amount of water or aqueous oxidizing composition can provide the same or greater lightening at the same or even lower strengths of oxidizing agents.

### Example 3C

A further study was performed where each of hair lightening compositions 2A-1, 2C-1, 2D-1, 2E-1, and 2F-1 was applied to separate swatches of natural level 4 hair (5 swatches total) at a rate of about 10 grams per gram of hair and covered with foil. The swatches treated with 2A-1, 2D-1, 2E-1, and 2F-1 were left to process at 33° C. for 35 minutes, and the swatch treated with 2C-1 was left to process at 33° C. for 50 minutes. The swatches were then rinsed, shampooed with a commercial shampoo, rinsed again, and blow dried. The change in lightness (ΔL) and tone (Δb) of the treated hair was then evaluated using Colorshot MS, as described above.

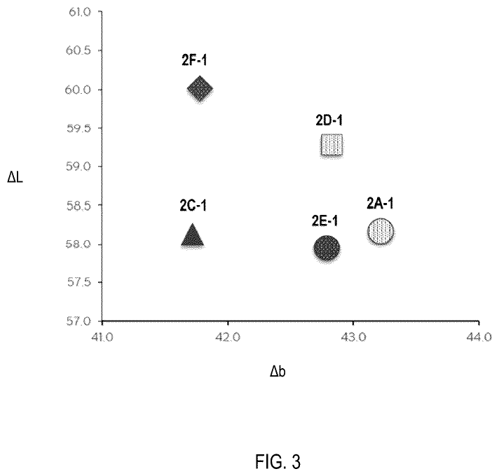

The results are shown in FIG. 3. As can be seen, all of the hair lightening compositions according to the disclosure provided equal or greater levels of lightening (ΔL) compared to traditional hair lightening compositions at the same strength (volume) of developer and over a shorter period of time. This study therefore demonstrates that the same very high level of lightening can be achieved in less time with hair lightening compositions according to the disclosure.

### Example 3D

A study was performed where each of hair lightening compositions 2A-2, 2A-4, 2C-1, 2D-2, 2F-2, and 2F-3 was applied to separate swatches of natural level 4 hair (6 swatches total) at a rate of about 10 grams per gram of hair and covered with foil. The swatches were left to process at 33° C. for 50 minutes, rinsed, shampooed with a commercial shampoo, rinsed again, and blow dried. The change in the lightness (ΔL) and tone (Δb) of the treated hair was then evaluated using Colorshot MS, as described above.

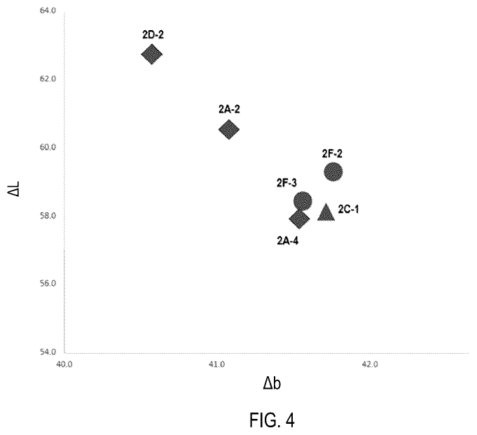

The results are shown in FIG. 4. As can be seen, even at lower strengths (volume) of developer, when processed for the same amount of time under the same conditions, hair lightening compositions according to the disclosure provided about the same or even significantly increased levels of lightening (ΔL) and tone (Δb) compared to traditional hair lightening compositions.

These results demonstrate that reducing the amount of damaging persulfate compounds and oxidizing agents surprisingly results in the same or even greater lightening effects over the same period of time.

### Example 3E

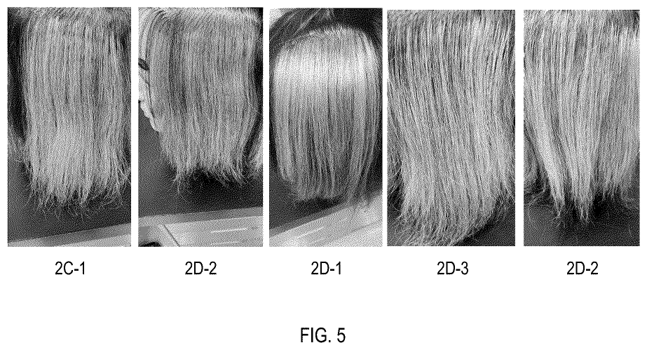

A study was performed on mannequin heads. Each of hair lightening compositions 2C-1, 2D-2, and 2D-3 were applied to mannequin heads with natural level 4 hair at a rate of about 10 grams per gram of hair and covered with foil, and the hair was left to process at 33° C. for 50 minutes. Separately, each of hair lightening compositions 2D-1 and 2D-2 were applied to mannequin heads with natural level 4 hair at a rate of about 10 grams per gram of hair and covered with foil, and the hair was left to process at 33° C. for 35 minutes. Once processing was complete, the hair was rinsed, shampooed with a commercial shampoo, rinsed again, and blow dried. The change in the color of the treated hair was then evaluated visually. The results of the study can be seen in FIG. 5.

Surprisingly, the results of this study showed that (1) at lower strengths (volume) of developer, when processed for the same amount of time under the same conditions, and (2) at the same strength of developer but for a shorter amount of time under the same conditions, hair lightening compositions according to the disclosure provided the same or even higher levels of lightening compared to traditional hair lightening compositions.

### Example 4—Evaluation of Properties of Treated Hair

In order to evaluate the properties of hair treated with lightening compositions according to the disclosure, compared to traditional hair lightening compositions, the following studies were conducted. In these studies, SEM images were obtained using a Hitachi TM4000-Plus scanning electron microscope. In each study, 8 hair fibers were selected randomly and 5 locations were imaged per hair fiber (40 total) to obtain statistical results.

### Example 4A

A study was performed where hair lightening composition 2C-7 was applied to three separate swatches of natural level 4 hair at a rate of about 10 grams per gram of hair and covered with foil. The swatches were left to process at 33° C. for 35 (swatch S1), 50 (swatch S2), or 70 (swatch S3) minutes. The swatches were then rinsed, shampooed with a commercial shampoo, rinsed again, and blow dried.

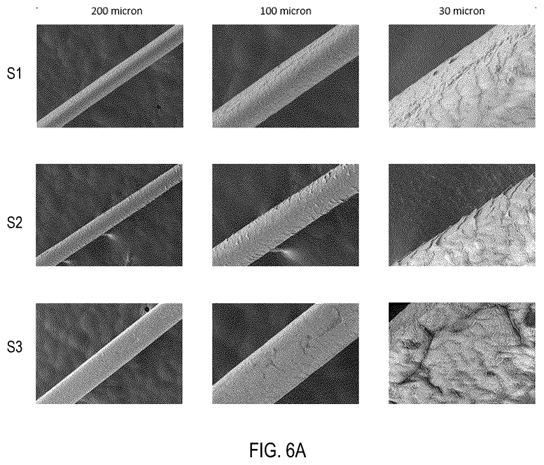

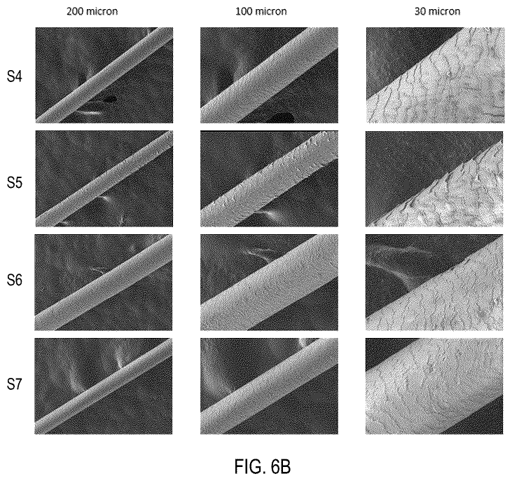

Once dry, 8 hair fibers were selected randomly from each swatch, and SEM images were obtained from 5 different locations per hair fiber (40 total) at 200 microns, 100 microns, and 30 microns. The images (FIG. 6A) obtained of S1 show minimal cuticle lifting or damage, while the images obtained of S2 show increased cuticle lifting and the images obtained of S3 show cracked and damaged hair fibers. The images shown in FIG. 6A represent the average seen from each image of S1-S3.

This study shows that traditional hair lightening compositions leave hair fibers with lifted cuticles and damaged hair fibers, which increases with increased processing time, such as may be required to achieve higher levels of lightening. The lifted cuticles and damaged fibers leave the hair feeling rough and unhealthy.

### Example 4B

Further studies were performed where each of hair lightening compositions 2C-7, 2F-1, and 2G-1 was applied to swatches of natural level 4 hair at a rate of about 10 grams per gram of hair, covered with foil, and left to process at 33° C. for 50 minutes. The swatches were then rinsed, shampooed with a commercial shampoo, rinsed again, and blow dried.

Once dry, 8 hair fibers were selected randomly from an untreated swatch (S4), the swatch treated with 20-7 (S5), the swatch treated with 2F-1 (S6), and the swatch treated with 2G-1 (S7), and SEM images were obtained from 5 different locations per hair fiber (40 total) at 200 microns, 100 microns, and 30 microns. The images (FIG. 6B) obtained of S4 show no cuticle lifting or damage, the images obtained of S5 show cuticle lifting, while the images obtained of S6 and S7 show almost no cuticle lifting or damage to the fibers. The images shown in FIG. 6B represent the average seen from each image of S4-S7.

FIG. 6B demonstrates that hair treated with hair lightening compositions according to the disclosure has smoother and less damaged hair fibers than hair treated with traditional hair lightening compositions having higher amounts of persulfate compounds. In addition, swatches S6 and S7 felt smoother and appeared healthier than swatch S5.

The results in this Example therefore demonstrate that hair treated with hair lightening compositions according to the disclosure has improved cosmetic properties, such as smoothness and healthy look and feel, compared to hair treated with traditional hair lightening compositions.

### Example 5—Multiple-Application Method of Lightening Hair

In order to evaluate the benefits achieved with a multiple-application method compared to a single-application method, the following study was performed.

Single application: Each of hair lightening compositions 2C-4, 2C-1, 2D-3, 2D-2, and 2D-1 was applied to swatches of natural level 4 hair (swatches S1-S5) at a rate of about 3 grams per gram of hair, covered with foil, and left to process at 33° C. for 50 minutes. The swatches were then rinsed, shampooed with a commercial shampoo, rinsed again, and blow dried.

Multiple application: Each of hair lightening compositions 2C-1, 2D-3, 2D-2, and 2D-1 was applied to swatches of natural level 4 hair (swatches S6-S9) at a rate of about 3 grams per gram of hair, covered with foil, and left to process at 33° C. for 25 minutes. The foils were removed and the swatches were wiped to remove substantially all of the hair lightening composition, and each swatch was treated with a second application of the same composition as used in the first application to the same swatch. The swatches were again covered with foil and left to process at 33° C. for 25 minutes, and then the swatches were rinsed, shampooed with a commercial shampoo, rinsed again, and blow dried.

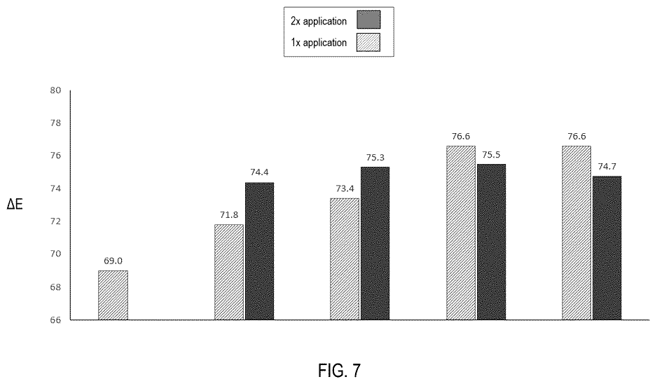

Once the swatches were dry, the change in the color of the hair relative to the color of the untreated natural level 4 hair was evaluated with the CIE L*a*b* system using Colorshot MS. The results are shown in Table 4 and FIG. 7.

TABLE 4
Single Application

| | Treatment | | Processing | | |
|---|---|---|---|---|---|
| Swatch | Composition | Developer | Time | ΔE | ΔL |
| S8 | 2C-4 | 20 V | 50 minutes | 69.0 | 53.1 |
| S9 | 2C-1 | 40 V | 50 minutes | 71.8 | 57.5 |
| S10 | 2D-3 | 20 V | 50 minutes | 73.4 | 60.0 |
| S11 | 2D-2 | 30 V | 50 minutes | 76.6 | 62.0 |
| S12 | 2D-1 | 40 V | 50 minutes | 76.6 | 65.1 |

Multiple Application

| | | | First | Second | | |
|---|---|---|---|---|---|---|
| | Treatment | | Processing | Processing | | |
| Swatch | Composition | Developer | Time | Time | ΔE | ΔL |
| S13 | 2C-1 | 40 V | 25 mins | 25 mins | 74.4 | 61.6 |
| S14 | 2D-3 | 20 V | 25 mins | 25 mins | 75.3 | 61.7 |
| S15 | 2D-2 | 30 V | 25 mins | 25 mins | 75.5 | 63.5 |
| S16 | 2D-1 | 40 V | 25 mins | 25 mins | 74.7 | 62.2 |

As can be seen in Table 4 and FIG. 7, with 30V and 40V developers, hair lightening compositions according to the disclosure provided ultra-high lift with either a single (S11, S12) or multiple (S15, S16) applications. However a second application was needed to achieve of the same level of lift with hair lightening composition according to the disclosure with a 20V developer (S14), while the comparative hair lightening composition required twice as much oxidizing agent (40V) to achieve the same level of lift using the same procedure (S13).

### Example 6—Additional Studies

Further studies to evaluate the lightening efficacy of hair lightening compositions having reduced amounts of persulfates were conducted.

Base lightening compositions 3A-3E according to the disclosure and comparative base lightening compositions C2-C4 were prepared shown in Tables 5A-5B.

TABLE 5A

| | 3A | 3B | 3C | 3D | 3E |
|---|---|---|---|---|---|
| UREA | 4.00 | 6.00 | 6.00 | 4.50 | 6.00 |
| POTASSIUM CARBONATE | 5.00 | | 2.70 | 5.00 | 4.00 |
| SODIUM SILICATE | 34.00 | 32.00 | 30.00 | 34.00 | 33.00 |
| SODIUM METASILICATE | 6.00 | 5.00 | 3.00 | 5.00 | 5.00 |
| SODIUM PERSULFATE | | 13.00 | | | 11.00 |
| DISODIUM EDTA | 1.20 | 1.20 | 1.20 | 1.19 | 1.19 |
| GLYCINE | 2.50 | 2.50 | 2.50 | 2.50 | 2.50 |
| SODIUM CITRATE | 3.00 | 4.50 | 6.00 | 3.25 | 3.50 |
| AMMONIUM PERSULFATE | 10.00 | 11.00 | 11.00 | 10.00 | 11.00 |
| POTASSIUM PERSULFATE | 25.00 | 14.00 | 27.00 | 25.00 | 15.00 |
| ACRYLATES/C10-30 ALKYL | 1.45 | 1.70 | 1.70 | 1.70 | 1.70 |
| ACRYLATE CROSSPOLYMER | | | | | |
| HYDROXYETHYLCELLULOSE | 2.70 | 2.10 | 2.70 | 2.70 | 2.60 |
| SODIUM LAURYL SULFATE | 1.20 | 1.20 | 1.20 | 1.19 | 1.19 |
| SODIUM STEARATE | 1.60 | 3.30 | 2.50 | 1.60 | |
| MINERAL OIL | 2.35 | 2.50 | 2.50 | 2.35 | 2.30 |
| TOTAL PERSULFATES | 35.00 | 38.00 | 38.00 | 35.00 | 37.00 |

TABLE 5B

| | C2 | C3 | C4 |
|---|---|---|---|
| UREA | | 2.50 | 2.00 |
| SODIUM SILICATE | 17.36 | 4.37 | 28.00 |
| SODIUM METASILICATE | 2.48 | 14.50 | 1.50 |
| DISODIUM EDTA | 0.98 | 0.95 | 1.00 |
| GLYCINE | | 2.00 | 2.00 |
| SODIUM CITRATE | 0.01 | | |
| CITRIC ACID | | 3.64 | 3.00 |
| AMMONIUM PERSULFATE | 4.96 | 11.60 | 11.00 |
| POTASSIUM PERSULFATE | 49.61 | 41.60 | 45.70 |
| ACRYLATES/C10-30 ALKYL ACRYLATE | | | 1.90 |
| CROSSPOLYMER | | | |
| HYDROXYETHYLCELLULOSE | | | 1.90 |
| GUAR GUM | 2.08 | 1.99 | |
| SODIUM LAURYL SULFATE | 1.10 | 0.80 | 1.00 |
| SODIUM STEARATE | 9.92 | 3.00 | |
| MINERAL OIL | 1.98 | 4.00 | |
| MAGNESIUM CARBONATE HYDROXIDE | 9.43 | 9.00 | |
| HYDROGENATED POLYDECENE | | | 1.00 |
| TOTAL PERSULFATES | 54.57 | 53.20 | 56.70 |

Hair lightening compositions 4A-4E and C5-C7 were prepared by mixing one of base lightening compositions 3A-3E according to the disclosure or comparative base lightening compositions C2-C4 with developer composition D1 of Table 1C just before use, as shown in Table 6.

TABLE 6

| Hair Lightening | Base Lightening | | |
|---|---|---|---|
| Composition | Composition | Developer | Mix Ratio |
| 4A | 3A | D2 | 1:2 |
| 4B | 3B | D2 | 1:2 |
| 4C | 3C | D2 | 1:2 |
| 4D | 3D | D2 | 1:2 |
| 4E | 3E | D2 | 1:2 |
| C5 | C2 | D2 | 1:1.5 |
| C6 | C3 | D2 | 1:1.5 |
| C7 | C4 | D2 | 1:1.5 |

Hair lightening composition 4F according to the disclosure was prepared by mixing 89 grams of composition 1D from Table 1A with 11 grams of water to form a base lightening mixture, and then mixing the base lightening mixture with developer composition D2 at a mix ratio of [1D+water]:developer of 1:2.

### Example 6A—Single Application

Hair lightening compositions 4A, 4D, and C6 were applied to separate swatches of straight, natural level 4 hair at a rate of about 10 grams per gram of hair and covered with foil. The swatches were left to process at 33° C. for 50 minutes, rinsed, shampooed with a commercial shampoo, rinsed again, and blow dried.

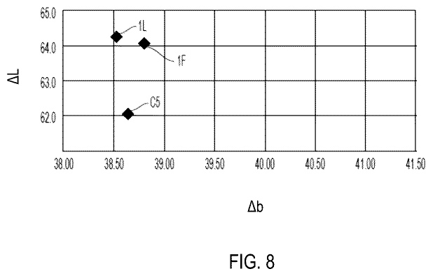

The change in lightness (ΔL) and tone (Δb) of the treated hair was then evaluated using ColorShot MS, as described above. The results are shown in FIG. 8. As can be seen, hair lightening compositions 4A and 4D according to the disclosure achieve better lightening than comparative hair lightening composition C6. Notably, because a ΔL greater than 2 is generally visible to the naked eye, this increased lift was noticeable.

### Example 6B—Multiple Application

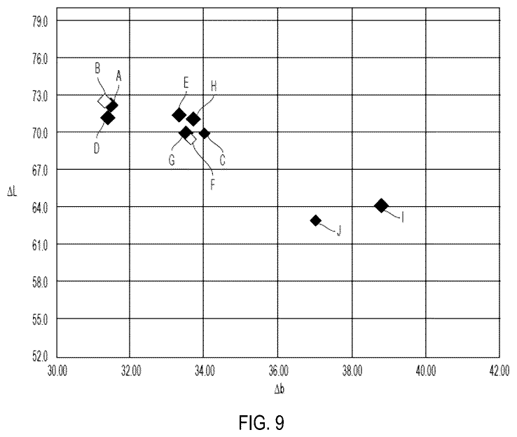

The hair lightening compositions from Table 4 were applied to separate swatches of natural level 4 hair at a rate of about 10 grams per gram of hair and covered with foil. The swatches were left to process at 33° C. for the period of time indicated for the first processing time in Table 7, rinsed, shampooed with a commercial shampoo, rinsed again, and blow dried. The process was repeated with each swatch being left to process at 33° C. for the period of time indicated for the second processing time in Table 7, after which the swatch was rinsed, shampooed, rinsed again, and blow dried. The change in lightness (ΔL) and tone (Δb) of the treated hair was measured after the second treatment using ColorShot MS, as described above. The results are shown in FIG. 9.

TABLE 7

| | Hair Lightening | First | Second |
|---|---|---|---|
| Swatch | Composition | Processing Time | Processing Time |
| A | 4D | 50 | 50 |
| B | C6 | 50 | 50 |
| C | 4D | 40 | 40 |
| D | C5 | 50 | 50 |
| E | 4F | 40 | 40 |
| F | 4C | 50 | 50 |
| G | 4E | 50 | 50 |
| H | 4B | 50 | 50 |
| I | 4F | 35 | 35 |
| J | C7 | 50 | 50 |

As FIG. 9 shows, multiple applications of hair lightening compositions according to the disclosure achieve the same or better lightening than comparative hair lightening compositions, under comparable conditions.

The above Examples demonstrate that base lightening compositions, base lightening mixtures, and hair lightening compositions according to the disclosure surprisingly provide very high and even ultra-high levels of hair lightening despite containing a reduced amount of persulfate compounds relative to traditional hair lightening compositions, in the same amount of time or less time. This reduction in persulfate compounds without extending the time needed to achieve the desired lightening also makes it possible to reduce hair damage and maintain hair fiber integrity, resulting in smoother, healthier looking and feeling hair compared to hair treated with traditional hair lightening compositions.

1. A method of lightening hair comprising:
(I) preparing a first hair lightening composition by mixing:
(a) a hair base lightening composition comprising:
(i) ammonium persulfate;
(ii) at least one additional persulfate compound; and
(iii) at least one silicate compound,
wherein the total amount of persulfate compounds in the hair base lightening composition ranges from about 10% to not greater than 50% by weight, relative to the total weight of the hair base lightening composition, and
wherein the weight ratio of (a)(i):(a)(ii) is less than 1;
(b) at least one solvent,
wherein the total amount of solvent ranges from about 1% to about 25% by weight, relative to the total weight of the hair base lightening composition; and
(c) at least one oxidizing agent,
(II) applying the first hair lightening composition to the hair,
(III) removing the first hair lightening composition from the hair,
(IV) applying a second hair lightening composition to the hair, and
(V) removing the second hair lightening composition from the hair,
wherein the first and second hair lightening compositions are applied to the hair within less than about hours of each other.

2. The method according to claim 1, wherein the total amount of persulfate compounds in the hair base lightening composition ranges from about 25% to about 45% by weight, relative to the total weight of the hair base lightening composition.

3. The method according to claim 1, wherein the weight ratio of (a)(i):(a)(ii) in the hair base lightening composition ranges from about 0.1 to about 0.9.

4. The method according to claim 1, wherein the hair base lightening composition comprises (a) (iii) at least one metasilicate compound.

5. The method according to claim 1, wherein the total amount of (b) solvent ranges from about 5% to about 20% by weight, relative to the total weight of the base lightening composition.

6. The method according to claim 1, wherein the weight ratio of the total amount of persulfates in the hair base lightening composition to the total amount of solvent ranges from about 1:1 to about 10:1.

7. The method according to claim 1, wherein the first and second hair lightening compositions are the same.

8. The method according to claim 1, wherein:
the first hair lightening composition is left on the hair to process for a first processing time,
the second hair lightening compositions is left on the hair to process for a second processing time, and
the first and second processing times independently range from about 15 minutes to about 50 minutes.

9. The method according to claim 1, wherein step (III) is performed by a method other than rinsing the first hair lightening composition from the hair.

10. The method according to claim 1, comprising, in order:
(A) mixing (a) and (b) to form a base lightening mixture, where (b) comprises water,
(B) mixing the base lightening mixture and (c) to form the first hair lightening composition,
(C) applying the first hair lightening composition to the hair,
(D) allowing the first hair lightening composition to process on the hair for a first processing time ranging from about 15 minutes to about 50 minutes,
(E) removing the first hair lightening composition from the hair,
(F) applying the second hair lightening composition to the hair, wherein the second hair lightening composition is the same as the first hair lightening composition,
(G) allowing the second hair lightening composition to process on the hair for a second processing time ranging from about 15 minutes to about 50 minutes, and
(H) rinsing and/or washing the hair.

11. The method according to claim 10, wherein the total amount of (b) solvent ranges from about 5% to about 20% by weight, relative to the total weight of the base lightening composition.

12. The method according to claim 10, wherein the weight ratio of the total amount of persulfates in the base lightening composition to the total amount of solvent ranges from about 1:1 to about 10:1.

13. The method according to claim 10, wherein step (E) is performed by a method other than rinsing the first hair lightening composition from the hair.

14. The method according to claim 1, further comprising heating the hair to which the first and/or second hair lightening compositions have been applied.

15. The method according to claim 1, comprising, in order:
(A) applying the second hair lightening composition to the hair,
(B) allowing the second hair lightening composition to process on the hair for a first processing time ranging from about 15 minutes to about 50 minutes,
(C) removing the second hair lightening composition from the hair,
(D) applying the first hair lightening composition to the hair, wherein the first hair lightening composition is different than the second hair lightening composition,
(E) allowing the first hair lightening composition to process on the hair for a second processing time ranging from about 15 minutes to about 50 minutes, and
(F) rinsing and/or washing the hair.

16. A method of lightening hair comprising:
(I) preparing a first hair lightening composition by mixing:
(a) a hair base lightening composition comprising:
(i) ammonium persulfate;
(ii) at least one additional persulfate compound; and
(iii) at least one silicate compound,
wherein the total amount of persulfate compounds in the hair base lightening composition ranges from about 10% to not greater than 50% by weight, relative to the total weight of the hair base lightening composition, and
wherein the weight ratio of (a)(i):(a)(ii) is less than 1; and
(b) an aqueous oxidizing composition,
wherein the effective mix ratio of (a):(b) ranges from about 1:3 to about 3:1,
(II) applying the first hair lightening composition to the hair,
(III) removing the first hair lightening composition from the hair,
(IV) applying a second hair lightening composition to the hair, and
(V) removing the second hair lightening composition from the hair,
wherein the first and second hair lightening compositions are applied to the hair within about 24 hours of each other.

17. The method according to claim 16, wherein the weight ratio of (a)(i):(a)(ii) in the hair base lightening composition ranges from about 0.1 to about 0.9.

18. The method according to claim 16, wherein the hair base lightening composition comprises (a) (iii) at least one metasilicate compound.

19. The method according to claim 16, wherein the first and second hair lightening compositions are the same.

20. A method of achieving ultra-high lightening of hair comprising:
(I) preparing a hair lightening composition by mixing:
(a) a hair base lightening composition comprising:
(i) ammonium persulfate;
(ii) at least one additional persulfate compound; and
(iii) at least one silicate compound,
wherein the total amount of persulfate compounds in the hair base lightening composition ranges from about 10% to not greater than 50% by weight, relative to the total weight of the hair base lightening composition, and
wherein the weight ratio of (a)(i):(a)(ii) is less than 1;
(b) a solvent, and
(c) an aqueous oxidizing composition comprising at least about 9% hydrogen peroxide by weight, relative to the total weight of the oxidizing composition,
(II) applying the hair lightening composition to the hair, and
(III) removing the hair lightening composition from the hair after a desired processing time,
wherein the hair has ultra-high lift after step (III) relative to before step (II).
